(19) United States
(12) Patent Application Publication
(10) Pub. No.: US 2002/0065221 A1
(43) Pub. Date: May 30, 2002

(54) Control of protein synthesis, and screening method for agents

(76) Inventors: Philip Cohen, Dundee (GB); Dario Alessi, Dundee (GB); Darren Cross, Dundee (GB)

Correspondence Address: Braman & Rogalskyj, LLP, P.O. Box 352, Canandaigua, NY, 14424-0352, US

(21) Appl. No.: 09/845,667

(22) Filed: Apr. 30, 2001

Related U.S. Application Data
(63) Continuation of application No. 09/091,763, filed on Jun. 19, 1998, now abandoned.
Application No. 09/091,763 is a 371 of international application No. PCT/GB96/03186, filed on Dec. 20, 1996.

(30) Foreign Application Priority Data
Jul. 18, 1996 (GB) 9615066.9
May 16, 1996 (GB) 9610272.8
Dec. 20, 1995 (GB) 9526083.2

(51) Int. Cl. A61K 38/52; A61K 38/51; A61K 38/53

(52) U.S. Cl. 514/12; 424/94.5

(57) ABSTRACT

A method for screening for agents capable of affecting the activity of kinases GSK3 and PKB is disclosed. The method involves assessing the phosphorylation of PKB on two amino acids on the PKB molecule particularly.

The present invention relates to the control of glycogen metabolism and protein synthesis, in particular through the use of insulin.

Many people with diabetes have normal levels of insulin in their blood, but the insulin fails to stimulate muscle cells and fat cells in the normal way (type II diabetes). Currently it is believed that there is a breakdown in the mechanism through which insulin signals to the muscle and fat cells.

The enzyme glycogen synthase kinase-3 (GSK3) (Embi et al., 1980) is implicated in the regulation of several physiological processes, including the control of glycogen (Parker et al., 1983) and protein (Welsh et al., 1993) synthesis by insulin, modulation of the transcription factors AP-1 and CREB (Nikolaki et al, de Groot et al., 1993 and Fiol et al 1994), the specification of cell fate in Drosophila (Siegfied et al., 1992) and dorsoventral patterning in Xenopus embryos (He et al., 1995). GSK3 is inhibited by serine phosphorylation in response to insulin or growth factors (Welsh et al., 1993, Hughes et al., 1994, Cross et al., 1994 and Saito et al., 1994) and in vitro by either MAP kinase-activated protein (MAPKAP) kinase-1 (also known as p90^{rsk}) or P70 ribosomal S6 kinase (p70^{S6k}) (Sutherland et al., 1993 and Sutherland et al., 1994).

We have now found, however, that agents which prevent the activation of both MAPKAP kinase-1 and p70^{S6k} by insulin in vivo do not block the phosphorylation and inhibition of GSK3. Another insulin-stimulated protein kinase inactivates GSK3 under these conditions, and we demonstrate that it is the product of the proto-oncogene Akt (also known as RAC or PKB; herein referred to as “PKB”).

GSK3 is inhibited in response to insulin with a half time of two min, slightly slower than the half time for activation of PKBα (one min). Inhibition of GSK3 by insulin results in its phosphorylation at the same serine residue (serine 21) which is targeted by PKBα in vitro. Like the activation of PKBα, the inhibition of GSK3 by insulin is prevented by phosphatidyl inositol (PI-3) kinase inhibitors wortmannin and LY 294002. The inhibition of GSK3 is likely to contribute to the increase in the rate of glycogen synthesis (Cross et al., 1994) and translation of certain mRNAs by insulin (Welsh et al., 1994).

Two isoforms of PKB, termed PKBα (Coffer & Woodgett, 1991), PKBβ (Cheng et al., 1992) and PKBγ (Konishi et al., 1995) have been identified and characterised. PKBβ, also known as RACβ and Akt-2, is over-expressed in a significant number of ovarian (Cheng et al., 1992) and pancreatic (Cheng et al., 1996) cancers and is over-expressed in the breast cancer epithelial cell line MCF7. PKB is composed of an N-terminal pleckstrin homology (PH) domain, followed by a catalytic domain and a short C-terminal tail. The catalytic domain is most similar to cyclic AMP-dependent protein kinase (PKA, 65% similarity) and to protein kinase C (PKC, 75% similarity) findings that gave rise to two of its names, namely PKB (i.e. between PKA and PKC) and RAC (Related to A and C kinase).

Many growth factors trigger the activation of phosphatidylinositol (PI) 3-kinase, the enzyme which converts PI 4,5 bisphosphate (PIP2) to the putative second messenger PI 3,4,5 trisphosphate (PIP3), and PKB lies downstream of PI 3-kinase (Franke et al., 1995). PKBα is converted from an inactive to an active form with a half time of about one minute when cells are stimulated with PDGF (Franke et al., 1995), EGF or basic FGF (Burgering & Coffer, 1995) or insulin (Cross et al., 1995 and Kohn et al., 1995) or perpervanadate (Andjelkovic et al., 1996). Activation of PKB by insulin or growth factors is prevented if the cells are preincubated with inhibitors of PI 3-kinase (wortmannin or LY 294002) or by overexpression of a dominant negative mutant of PI 3-kinase (Burgering & Coffer 1995). Mutation of the tyrosine residues in the PDGF receptor that when phosphorylated bind to PI 3-kinase also prevent the activation of PKBα (Burgering & Coffer, 1995 and Franke et al., 1995).

The present invention thus provides the use of PKB, its analogues, isoforms, inhibitors, activators and/or the functional equivalents thereof to regulate glycogen metabolism and/or protein synthesis, in particular in disease states where glycogen metabolism and/or protein synthesis exhibits abnormality, for example in the treatment of type II diabetes; also in the treatment of cancer, such as ovarian, breast and pancreatic cancer. A composition comprising such agents is also covered by the present invention, and the use of such a composition for treatment of disease states where glycogen metabolism and/or protein synthesis exhibit abnormality.

The present invention also provides a novel peptide comprising the amino acid sequence Arg-xaa-Arg-Yaa-zaa-Ser/Thr-Hyd, where Xaa is any amino acid, Yaa and Zaa are any amino acid (preferably not glycine), and Hyd is a large hydrophobic residue such as Phe or Leu, or a functional equivalent thereof. Represented in single letter code, a suitable peptide would be RXRX′X′S/TF/L, where X′ can be any amino acid, but is preferably not glycine; glycine can in fact be used, but other amino acids are preferred. Typical peptides include GRPRTSSFAEG, RPRAATC or functional equivalents thereof. The peptide is a substrate for measuring PKB activity.

The invention also provides a method for screening for substances which inhibit the activation of PKB in vivo by preventing its interaction with PIP3 or PI3,4-bisP.

Thus the invention also provides a method of determining the ability of a substance to affect the activity or activation of PKB, the method comprising exposing the substance to PKB and phosphatidyl inositol polyphosphate (ie PIP3, PI3,4-bisP etc) and determining the interaction between PKB and the phosphatidyl inositol polyphosphate. The interaction between PKB and the phosphatidyl inositol polyphosphate can conveniently be measured by assessing the phosphorylation state of PKB (preferably at T308 and/or S473), eg by measuring transfer of radiolabelled ³²P from the PIP3 (for example) to the PKB and/or by SDS-PAGE.

The method of the invention can also be used for identifying activators or inhibitors of GSK3, such a method can comprise exposing the substance to be tested to GSK3, and (optionally) a source of phosphorylation, and determining the state of activation of GSK3 (optionally by determining the state of its phosphorylation. This aspect of the invention can be useful for determining the suitability of a test substance for use in combatting diabetes, cancer, or any disorder which involves irregularity of protein synthesis or glycogen metabolism.

The invention also provides a method for screening for inhibitors or activators of enzymes that catalyse the phosphorylation of PKB, the method comprising exposing the substance to be tested to

one or more enzymes upstream of PKB;

PKB; and (optionally)

nucleoside triphosphate and determining whether (and optionally to what extent) the PKB has been phosphorylated on T308 and/or S473.

Also provided is a method of identifying agents able to influence the activity of GSK3, said method comprising:

a. exposing a test substance to a substrate of GSK3;

b. detecting whether (and, optionally, to what extent) said peptide has been phosphorylated.

The test substance may be an analogue, isoform, inhibitor, or activator of PKB, and the above method may be modified to identify those agents which stimulate or inhibit PKB itself. Thus such a method may comprise the following steps:

a. exposing the test substance to a sample containing PKB, to form a mixture;

b. exposing said mixture to a peptide comprising the amino acid sequence defined above or a functional equivalent thereof (usually in the presence of Mg²⁺ and ATP); and

c. detecting whether (and, optionally, to what extent) said peptide has been phosphorylated.

In this aspect the method of the invention can be used to determine whether the substance being tested acts on PKB or directly on GSK3. This can be done by comparing the phosphorylation states of the peptide and PKB; if the phosphorylation state of GSK3 is changed but that of PKB is not then the substance being tested acts directly on GSK3 without acting on PKB. In a further aspect the present invention provides a method of treatment of the human or non-human (preferably mammalian) animal body, said method comprising administering PKB, its analogues, inhibitors, stimulators or functional equivalents thereof to said body. Said method affects the regulation of glycogen metabolism in the treated body.

The method of treatment of the present invention may be of particular use in the treatment of type II diabetes (where desirably an activator of PKB is used, so that the down-regulation of GSK3 activity due to the action of PKB is enhanced).

The method of treatment of the present invention may alternatively be of particular use in the treatment of cancer such as ovarian cancer (where desirably an inhibitor of PKB is used, so that the down-regulation of GSK3 activity due to the action of PKB is depressed). Other cancers associated with irregularities in the activity of PKB and/or GSK3 may also be treated by the method, such as pancreatic cancer, and breast cancer.

Stimulation of PKB with insulin increases activity 12-fold within 5 min and induces its phosphorylation at Thr-308 and Ser-473. PKB transiently transfected into cells can be activated 20-fold in response to insulin and 46-fold in response to IGF-1 and also became phosphorylated at Thr-308 and Ser-473. The activation of PKB and its phosphorylation at both Thr-308 and Ser-473 can be prevented by the phosphatidylinositol (P1) 3-kinase inhibitor wortmannin. The phosphorylation of threonine 308 and serine 473 act synergistically to activate PKB.

MAPKAP kinase-2-phosphorylated PKB at Ser-473 in vitro increases activity seven-fold, an effect that can be mimicked (fivefold activation) by mutating Ser-473 to Asp. Mutation of Thr-308 to Asp also increases PKB activity five-fold and subsequent phosphorylation of Ser-473 by MAPKAP kinase-2 stimulates activity a further fivefold, an effect mimicked (18-fold activation) by mutating both Thr-308 and Ser-473 to Asp. The activity of the Asp-308/Asp-473 double mutant was similar to that of the fully phosphorylated enzyme and could not be activated further by insulin. Mutation of Thr-308 to Ala did not prevent the phosphorylation of transfected PKB at Ser-473 after stimulation of 293 cells with insulin or IGF-1, but abolished the activation of PKB. Similarly, mutation of Ser-473 to Ala did not prevent the phosphorylation of transfected PKB at Thr-308 but greatly reduced the activation of transfected PKB. This demonstrates that the activation of PKB by insulin or IGF-1 results from the phosphorylation of Thr-308 and Ser-473 and that phosphorylation of both residues is preferred to generate a high level of PKB activity in vitro or in vivo. Also, phosphorylation of Thr-308 in vivo is not dependent on the phosphorylation of Ser-473 or vice versa, that the phosphorylation of Thr-308 and Ser-473 are both dependent on PI 3-kinase activity and suggest that neither Thr-308 nor Ser-473 phosphorylation is catalysed by PKB itself.

Thus, it is preferred that the present invention incorporates the use of any agent which affects phosphorylation of PKB at amino acids 308 and/or 473, for example insulin, inhibitors of PI 3-kinase such as wortmannin or the like. The use of PKB, itself altered at amino acids 308 and/or 473 (eg by phosphorylation and/or mutation) is also suitable.

In a variation of the method of the present invention, stimulation or inhibition of PKB may be assessed by monitoring the phosphorylation states of amino acids 308 and/or 473 on PKB itself.

Different isoforms of PKB may be used or targeted in the present invention; for example PKBα, β or γ.

The present invention will now be described in more detail in the accompanying examples which are provided by way of non-limiting illustration, and with reference to the accompanying drawings.

### EXAMPLE 1

PKB Influences GSK3 Activity

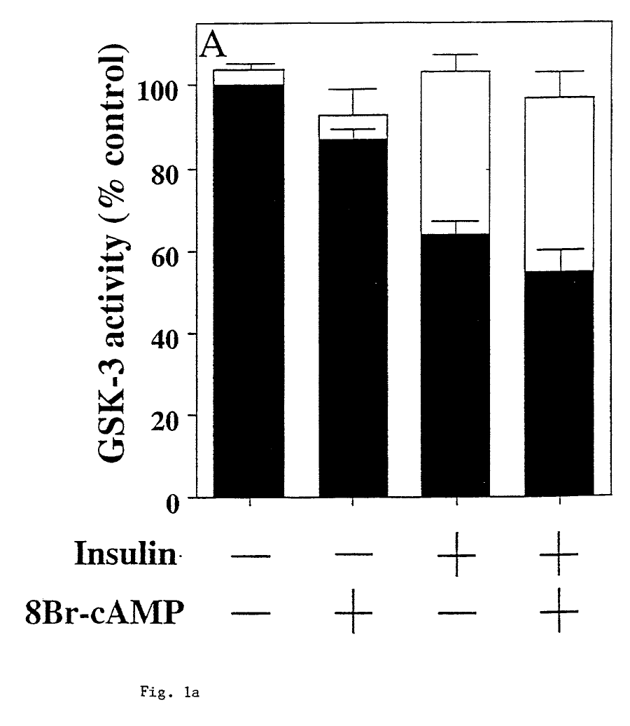

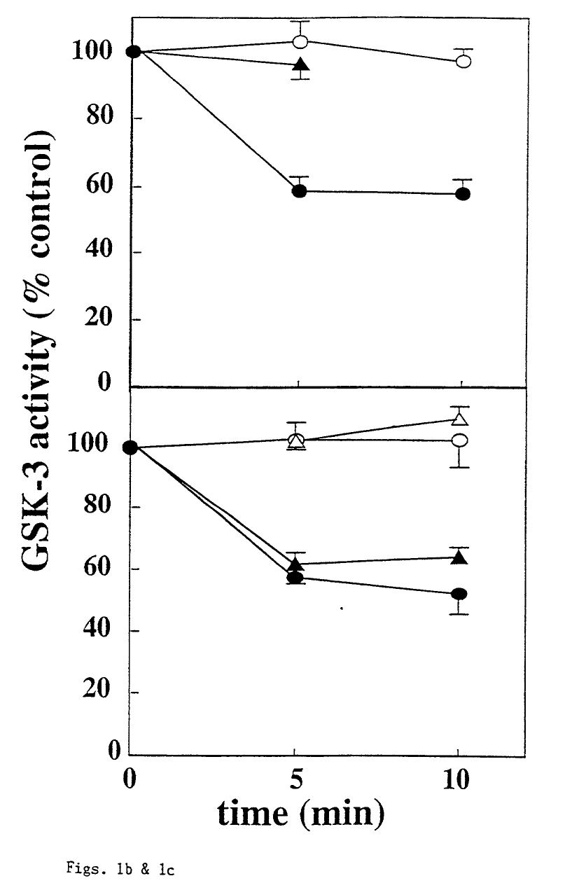

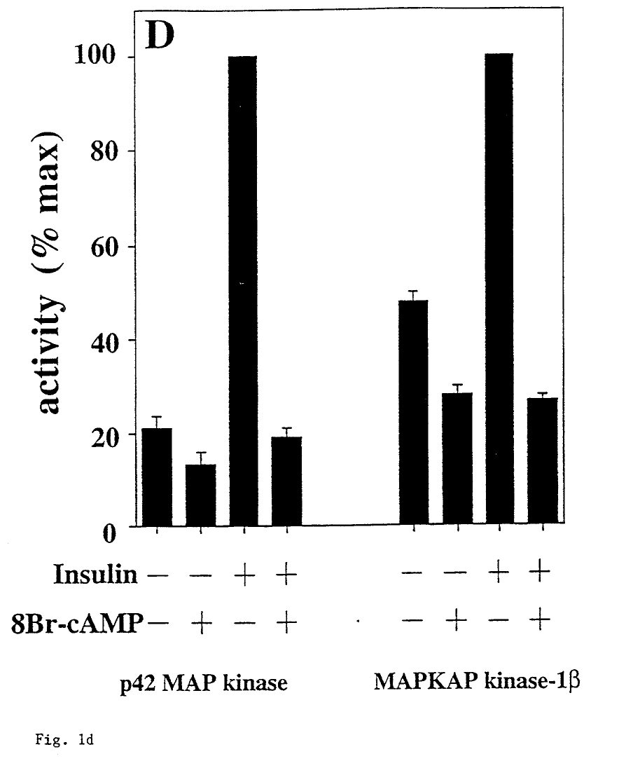

FIG. 1: a, L6 myotubes were incubated for 15 min with 2 mm 8-bromocyclic-AMP (8Br-cAMP) and then with 0.1 μM insulin (5 min). Both GSK3 isoforms were co-immunoprecipitated from the lysates and assayed before (black bars) and after (white bars) reactivation with PP2A (cross et al., 1994). The results are presented relative to the activity in unstimulated cells, which was 0.08±0.006 U mg⁻¹ (n=10).

b, c, The inhibition of GSK3 by insulin (0.1 μM) is unaffected by rapamycin (0.1 μM) and PD 98059 (50 μM), but prevented by LY 294002 (100 μM).

b, L6 myotubes were stimulated with insulin for the times indicated with (filled triangle) or without (filled circles) a 15 min preincubation with LY 294002, and GSK3 measured as in a. The open circles show experiments from insulin-stimulated cells where GSK3 was assayed after reactivation with PP2A (Cross et al., 1994).

c, cells were incubated with rapamycin (triangles) or rapamycin plus PD 98059 (circles) before stimulation with insulin, and GSK3 activity measured as in a, before (filled symbols) and after (open symbols) pretreatment with PP2A.

d, e, L6 myotubes were incubated with 8Br-cAMP (15 min) PD 98059 (60 min) or LY 294002 (15 min) and then with insulin (5 min) as in a-c. Each enzyme was assayed after immunoprecipitation from lysates, and the results are presented relative to the activities obtained. In the presence of insulin and absence of 8Br-cAMP, which were 0.04±0.005 U mg⁻¹ (p42 MAP kinase, n=6) and 0.071±0.004 U mg⁻¹ (MAPKAP Kinase⁻¹, n=6).

All the results (±s.e.m.) are for at least three experiments.

Monolayers of L6 cells were cultured, stimulated and lysed as described previously (Cross et al., 1994). p42 MAP kinase, MAPKAP kinase 1 or (GSK3-α plus GSK3-β) were then immunoprecipitated from the lysates and assayed with specific protein or peptide substrates as described previously (Cross et al., 1994). One unit of protein kinase activity was that amount which catalysed the phosphorylation of 1 nmol of substrate in 1 min. Where indicated, GSK3 in immunoprecipitates was reactivated with PP2A (Cross et al., 1994).

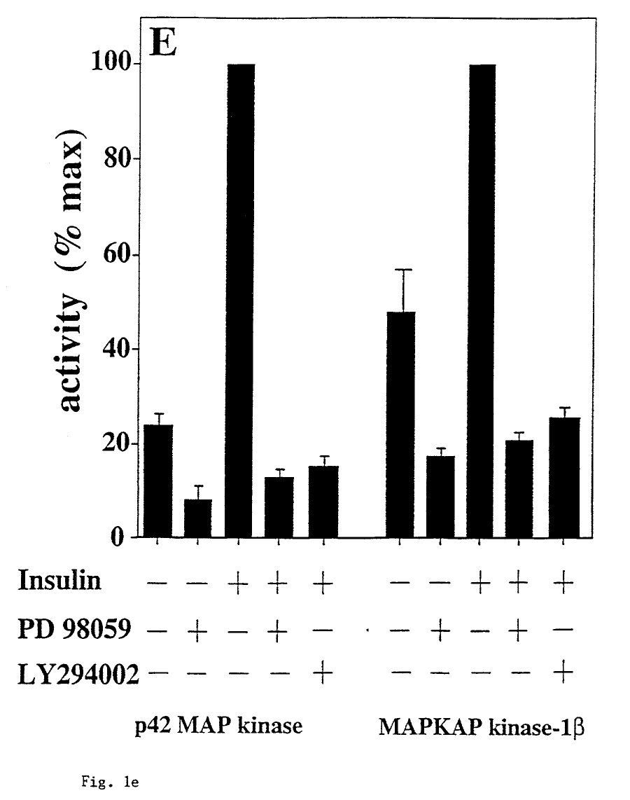

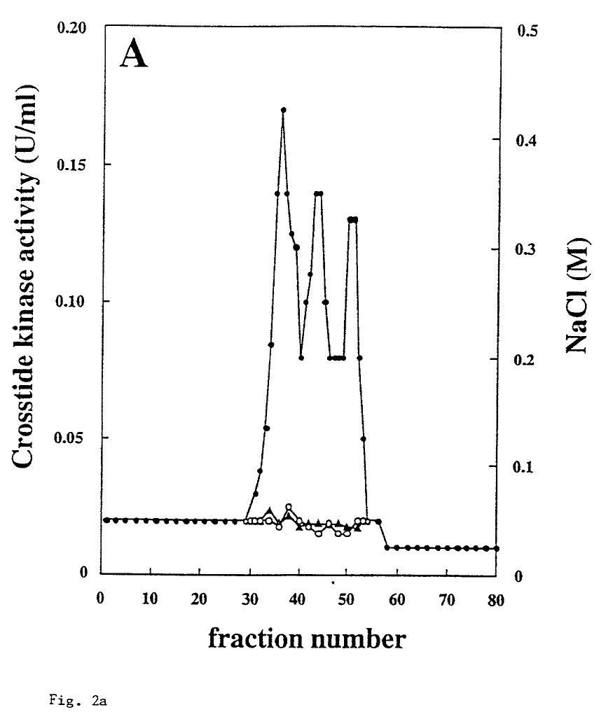

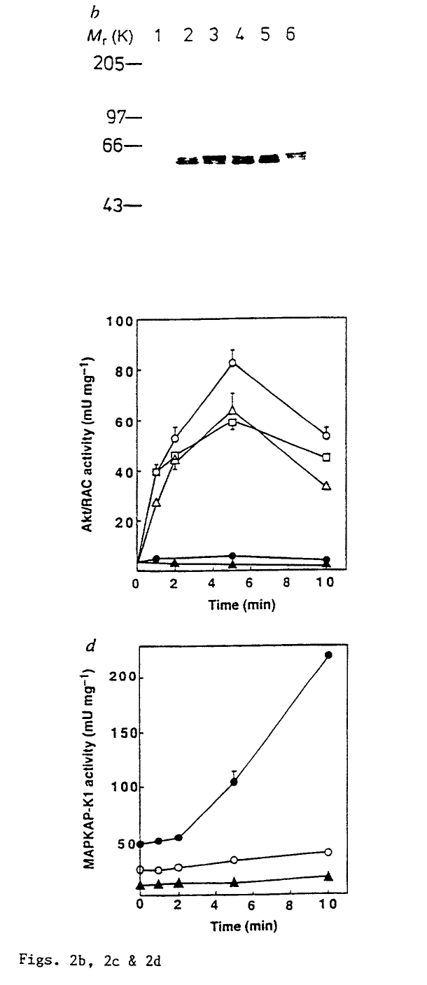

FIG. 2 Identification of PKB as the insulin-stimulated, wortmannin-sensitive and PD 98059/rapamycin-insensitive Crosstide kinase in L6 myotubes.

a. Cells were incubated with 50 μM PD 98059 (for 1 hour) and 0.1 μM rapamycin (10 min), then stimulated with 0.1 μM insulin (5 min) and lysed (Cross et al., 1994). The lysates (0.3 mg protein) were chromatographed on Mono Q (5×0.16 cm) and fractions (0.05 ml) were assayed for Crosstide kinase (filled circles). In separate experiments insulin was omitted (open circles) or wortmannin (0.1 μM) added 10 min before the insulin (filled triangles). The broken line shows the NaCl gradient. Similar results were obtained in six experiments.

b. Pooled fractions (10 μl) 31-34 (lane 1), 35-38 (lane 2), 39-42 (lane 3), 43-45 (lane 4), 46-49 (lane 5) and 50-53 (lane 6) from a were electrophoresed on a 10% SDS/polyacrylamide gel and immunoblotted with the C-terminal anti-PKBα antibody. Marker proteins are indicated. No immunoreactive species were present in fractions 1-30 or 54-80.

c. L6 myotubes were stimulated with 0.1 μM insulin and PKB immunoprecipitated from the lysates (50 μg protein) essentially as described previously (Lazar et al., 1995), using the anti-PH domain antibody and assayed for Crosstide kinase (open circles). In control experiments, myotubes were incubated with 0.1 μM rapamycin plus 50 μM PD 98059 (open triangles) or 2 mM 8Br-cAMP (open squares), or 0.1 μM wortmannin (filled circles) or 100 μM LY 294002 (filled triangles) before stimulation with insulin.

d. As c, except that MAPKAP kinase-1 was immunoprecipitated from the lysates and assayed with S6 peptide (filled circles). In control experiments, cells were incubated with 0.1 μM rapamycin plus 50 μM PD 98059 (filled triangles) or with 2 μM 8BR-cAMP (open circles) before stimulation with insulin. In c and d, the error bars denote triplicate determinations, and similar results were obtained in three separate experiments.

Mono Q chromatography was performed as described (Burgering et al., 1995), except that the buffer also contained 1 mM EGTA, 0.1 mM sodium orthovanadate and 0.5% (w/v) Triton X-100. Two PKBα antibodies were raised in rabbits against the C-terminal peptide FPQFSYSASSTA and bacterially expressed PH domain of PKBα. The C-terminal antibody was affinity purified (Jones et al., 1991). The activity of PKB towards Crosstide is threefold higher than its activity towards histone H2B and 11-fold higher than its activity towards myelin basic protein, the substrates used previously to assay PKB. Other experimental details and units of protein kinase activity are given in FIG. 1.

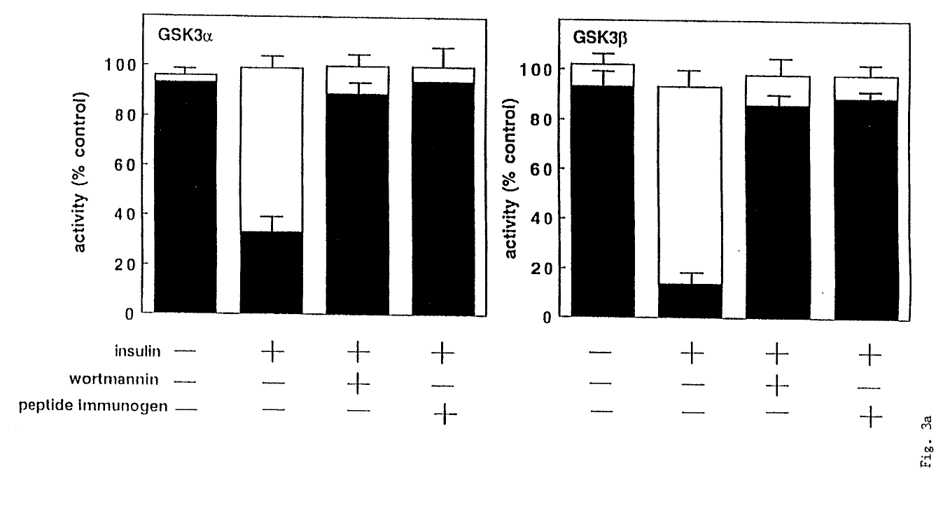

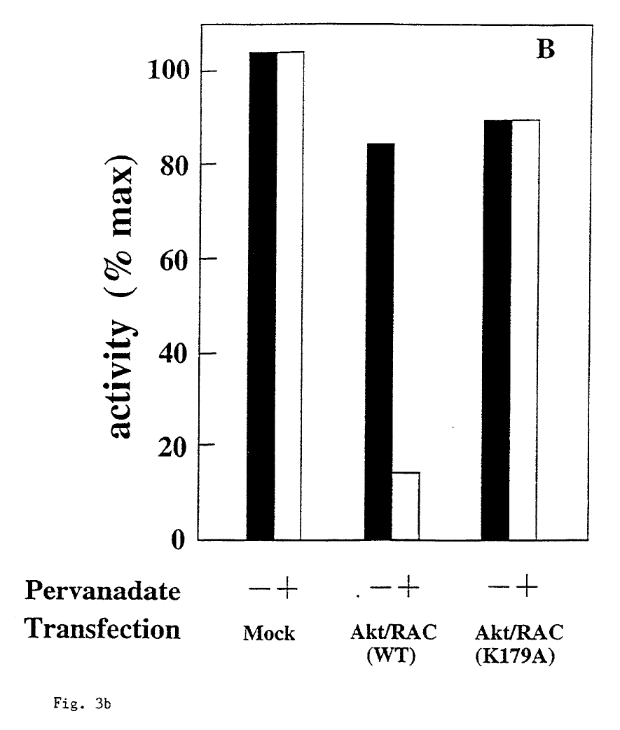

FIG. 3 GSK3 is inactivated by PKB from insulin-stimulated L6 myotubes.

a. Cells were stimulated for 5 min with 0.1 μM insulin, and PKB immunoprecipitated from 100 μg of cell lysate and used to inactivate GSK3 isoforms essentially as described previously (Sutherland et al., 1993 and Sutherland et al., 1994). The black bars show GSK3 activity measured after incubation with MgATP and PKB as a percentage of the activity obtained in control incubations where PKB was omitted. In the absence of PKB, GSK3 activity was stable throughout the experiment. The white bars show the activity obtained after reactivation of GSK3 with PP2A (Embi et al., 1980). No inactivation of GSK3 occurred if insulin was omitted, or if wortmannin (0.1 μM ) was added 10 min before the insulin, or if the anti-PKB antibody was incubated with peptide immunogen (0.5 mM) before immunoprecipitation. The results (±s.e.m.) are for three experiments (each carried out in triplicate).

b. Inactivation of GSK3-β by HA-PKBα. Complementary DNA encoding HA-PKBα was transfected into COS-1 cells, and after stimulation for 15 min with 0.1 mM sodium pervanadate the tagged protein kinase was immunoprecipitated from 0.3 mg of lysate and incubated for 20 min with GSK3-β and MgATP. In control experiments, pervanadate was omitted, or wildtype (WT) PKBα replaced by vector (mock translation) or by a kinase-inactive mutant of PKBα in which Lys 179 was mutated to Ala (K179A). Similar results were obtained in three separate experiments. The levels of WT and K179A-PKBα in each immunoprecipitate were similar in each transfection.

In a GSK3-α and GSK3-β were partially purified, assayed, inactivated by PKB, and reactivated by PP2A from rabbit skeletal muscle as described previously (Sutherland et al., 1993 and Sutherland et al., 1994). There was no reactivation in control experiments in which okadaic acid (2 μM ) was added before PP2A.

FIG. 4: Identification of the residues in GSK3 phosphorylated by PKB in vitro and in response to insulin in L6 myotubes.

a. GSK3-β was maximally inactivated by incubation with PKB and Mg-[γ-³²P]ATP and after SDS-PAGE, the ³²P-labelled GSK3-β (Mᵣ 47K) was digested with trypsin¹¹ and chromatographed on a C₁₈ column (Sutherland et al., 1993). Fractions (0.8 ml) were analysed for ³²P-radioactivity (open circles), and the diagonal line shows the acetonitrile gradient.

b. The major phosphopeptide from a (400 c.p.m.) was subjected to solid-phase sequencing (Sutherland et al., 1993), and ³²P-radioactivity released after each cycle of Edman degradation is shown.

c. GSK3-α and GSK3-β were co-immunoprecipitated from the lysates of ³²P-labelled cells, denatured in SDS, subjected to SDS-PAGE, transferred to nitrocellulose and autoradiographed (Saito et al., 1994). Lanes 1-3, GSK3 isoforms immunoprecipitated from unstimulated cells; lanes 4-6, GSK3 isoforms immunoprecipitated from insulin-stimulated cells.

d. GSK3 isoforms from c. were digested with trypsin, and the resulting phosphopeptides separated by isoelectric focusing (Saito et al., 1994) and identified by auto-radiography. Lanes 1 and 4 show the major phosphopeptide resulting from in vitro phosphorylation of GSK3-β by PKB and MAPKAP kinase-1, respectively; lanes 2 and 5, the phosphopeptides obtained from GSK3-β and GSK3-α, immunoprecipitated from unstimulated cells; lanes 3 and 6, the phosphopeptides obtained from GSK3-β and GSK3-α immunoprecipitated from cells stimulated for 5 min with 0.1 μM insulin; the arrow denotes the peptides whose phosphorylation is increased by insulin. The pI values of two markers, Patent Blue (2.4) and azurin (5.7) are indicated.

In a. PKBα was immunoprecipitated with the C-terminal antibody from the lysates (0.5 mg protein) of insulin-stimulated L6 myotubes and used to phosphorylate GSK-β¹². In c. three 10-cm diameter dishes of L6 myotubes were incubated for 4 hours in HEPES-buffered saline (Cross et al., 1994) containing 50 μM PD 98059, 100 nM rapamycin and 1.5 mCI ml^{-1 32}P-orthophosphate, stimulated for 5 min with insulin (0.1 μM ) or buffer, and GSK3 isoforms co-immunoprecipitated from the lysates as in FIG. 1.

Discussion

Inhibition of GSK3 induced by insulin in L6 myotubes (FIGS. 1a-c) was unaffected by agents which prevented the activation of MAPKAP kinase-1 (8-bromo-cyclic AMP, or PD 98059 (Alessi et al., 1995), (FIG. 1d, e) and/or p70^{S6k} (rapamycin (Kuo et al., 1992)) (Cross et al., 1994), suggesting that neither MAPKAP kinase-1 nor p70^{S6k} are essential for this process. However, the phosphorylation and inhibition of GSK3-β after phorbol ester treatment (Stambolic et al., 1994) is enhanced by coexpression with MAPKAP kinase 1 in HeLa S3 cells, whereas in NIH 3T3 cells the EGF-induced inhibition of GSK3-α and GSK3-β (Saito et al., 1994) is largely suppressed by expression of a dominant-negative mutant of MAP kinase kinase-1 (Elgar et al., 1995). MAPKAP kinase-1 may therefore mediate the inhibition of GSK3 by agonists which are much more potent activators of the classical MAP kinase pathway than is insulin.

To identify the insulin-stimulated protein kinase that inhibits GSK3 in the presence of rapamycin and PD 98059, L6 myotubes were incubated with both compounds and stimulated with insulin. The lysates were then chromatographed on Mono Q and the fractions assayed with “Crosstide” (GRPRTSSFAEG), a peptide corresponding to the sequence in GSK3 surrounding the serine (underlined) phosphorylated by MAPKAP kinase-1 and p70^{S6k} (Ser 21 in GSK3-α (Sutherland et al., 1994) and Ser 9 in GSK3-β (Sutherland et al 1993)). Three peaks of Crosstide kinase activity were detected, which were absent if insulin stimulation was omitted or if the cells were first preincubated with the PI 3-kinase inhibitor wortmannin (FIG. 2a). Wortmannin (Cross et al., 1994 and Welsh et al 1994), and the structurally unrelated PI 3-kinase inhibitor LY 294002 (ref 19); (FIG. 1b), both prevent the inhibition of GSK3 by insulin.

The protein kinases PKB-α, PKB-β and PKBγ are Ser/Thr-specific and cellular homologues of the viral oncogene v-akt (Coffer et al., 1991, Jones et al 1991, Ahmed et al 1995 and Cheng et al., 1992). These enzymes have recently been shown to be activated in NIH 3T3, Rat-1 or Swiss 3T3 cells in response to growth factors or insulin, activation being suppressed by blocking the activation of PI 3-kinase in different ways (Franke et al., 1995 and Burgering et al., 1995). All three peaks of Crosstide kinase (FIG. 2a), but no other fraction from Mono Q, showed the characteristic multiple bands of PKB (relative molecular mass, Mᵣ 58K, 59K or 60K) that have been observed in other cells, when immunoblotting was performed with an antibody raised against the carboxyl-terminal peptide of PKB-α (FIG. 2b). The more slowly migrating forms represent more highly phosphorylated protein, and are converted to the fastest migrating species by treatment with phosphatases. Phosphatase treatment also results in the inactivation of PKB (Burgering et al., 1995) and the complete loss of Crosstide kinase activity (data not shown). Of the Crosstide kinase activity in peaks 2 and 3 from Mono Q, 70-80% was immunoprecipitated by a separate antibody raised against the amino-terminal pleckstrin homology (PH) domain of PKB-α. The C-terminal antibody also immunoprecipitated PKB activity specifically from peaks 2 and 3, but was less effective than the anti-PH-domain antibody. Peak-1 was hardly immunoprecipitated by either antibody and may represent PKBβ. An immunoprecipitating anti-MAPKAP kinase-1 antibody (Cross et al., 1994) failed to deplete any of the Crosstide kinase activity associated with peaks 1, 2 or 3.

Insulin stimulation of L6 myotubes increased PKB activity by more than tenfold (FIG. 2c), and activation was blocked by wortmannin or LY 294002, but was essentially unaffected by 8-bromo-cyclic AMP or rapamycin plus PD 98059 (FIG. 2c). The half-time (t_{0.5}) or activation of PKB (1 min) was slightly faster than that for inhibition of GSK3 (2 min) (Cross et al., 1994). In contrast, the activation of MAPKAP kinase-1 (FIG. 2d) and p70^{S6k} (not shown) was slower (t_{0.5>5} min). Activation of MAPKAP kinase-1 was prevented by 8-bromo-cyclic AMP or PD 98059 (FIG. 2d), and activation of p70^{S6k} by rapamycin (Cross et al., 1994). Akt/RAC phosphorylated the Ser in the Crosstide equivalent to Ser 21 in GSK3-α and Ser 9 in GSK3-β (data not shown).

PKB from insulin-stimulated L6 myotubes (but not from unstimulated or wortmannin-treated cells) inactivated GSK3-α and GSK3-β in vitro, and inhibition was reversed by the Ser/Thr-specific protein phosphatase PP2A (Embi et al., 1980) (FIG. 3a). To further establish that inactivation was catalysed by PKB, and not by a co-immunoprecipitating protein kinase, haemagglutonin-tagged PKB-α (HA-PKB) was transfected into COS-1 cells and activated by stimulation with pervanadate, which is the strongest inducer of PKB activation in this system. The HA-PKB inactivated GSK3-β, but not if treatment with pervanadate was omitted or if wild-type HA-PKB was replaced with a “kinase inactive” mutant (FIG. 3b).

The inactivation of GSK3-β by PKB in vitro was accompanied by the phosphorylation of one major tryptic peptide (FIG. 4a) which coeluted during C₁₈ chromatography (Sutherland et al., 1993) and isoelectric focusing with that obtained after phosphorylation by MAPKAP kinase-1 (FIG. 4d). Stimulation of L6 myotubes with insulin (in the presence of rapamycin and PD 98059) increased the ³²P-labelling of GSK3-α and GSK3-β by 60-100% (FIG. 4c) and increased the ³²P-labelling of the same tryptic peptides labelled in vitro (FIG. 4d). Sequence analyses established that the third residue of these, corresponding to Ser 9 (GSK3-β) or Ser 21 (GSK3-α), was the site of phosphorylation in each phosphopeptide, both in vitro (FIG. 4b) and in vivo (not shown). The ³²P-labelling of other (more acidic) tryptic phosphopeptides was not increased by insulin (FIG. 4d). These peptides have been noted previously in GSK3 from A431 cells and shown to contain phosphoserine and phosphotyrosine (Saito et al., 1994).

PKC-δ, εand ζ are reported to be activated by mitogens, and PKC-ζ activity is stimulated in vitro by several inositol phospholipids, including PI(3, 4, 5)P₃ the product of the PI 3-kinase reaction (Andjelkovic et al., 1995). However, purified PKC-ε (Palmer et al., 1995), PKC-ε and PKC-ζ (data not shown) all failed to inhibit GSK3-α or GSK3-β in vitro. Moreover, although PKC-α, β1 and γ inhibit GSK3-β in vitro (Palmer et al., 1995), GSK3-α is unaffected, while their downregulation in L6 myotubes by prolonged incubation with phorbol esters abolishes the activation of MAPKAP kinase-1 in response to subsequent challenge with phorbol esters, but has no effect on the inhibition of GSK3 by insulin (not shown).

Taken together, our results identify GSK3 as a substrate for PKB. The stimulation of glycogen synthesis by insulin in skeletal muscle involves the dephosphorylation of Ser residues in glycogen synthase that are phosphorylated by GSK3 in vitro (Parker et al., 1983). Hence the 40-50% inhibition of GSK3 by insulin, coupled with a similar activation of the relevant glycogen synthase phosphatase (Goode et al., 1992), can account for the stimulation of glycogen synthase by insulin in skeletal muscle (Parker et al., 1983) or L6 myotubes (Goode et al., 1992). The activation of glycogen synthase and the resulting stimulation of glycogen synthesis by insulin in L6 myotubes is blocked by wortmannin, but not by PD 98059 (Dent et al., 1990), just like the activation of Akt/RAC and inhibition of GSK3. However, GSK3 is unlikely to be the only substrate of PKB in vivo, and identifying other physiologically relevant substrates will be important because PKBβ is amplified and over-expressed in many ovarian neoplasms (Cheng et al., 1992).

### EXAMPLE 2

Activation of PKB by Insulin in L6 Myotubes is Accompanied by Phosphorylation of Residues Thr-308 and Ser-473

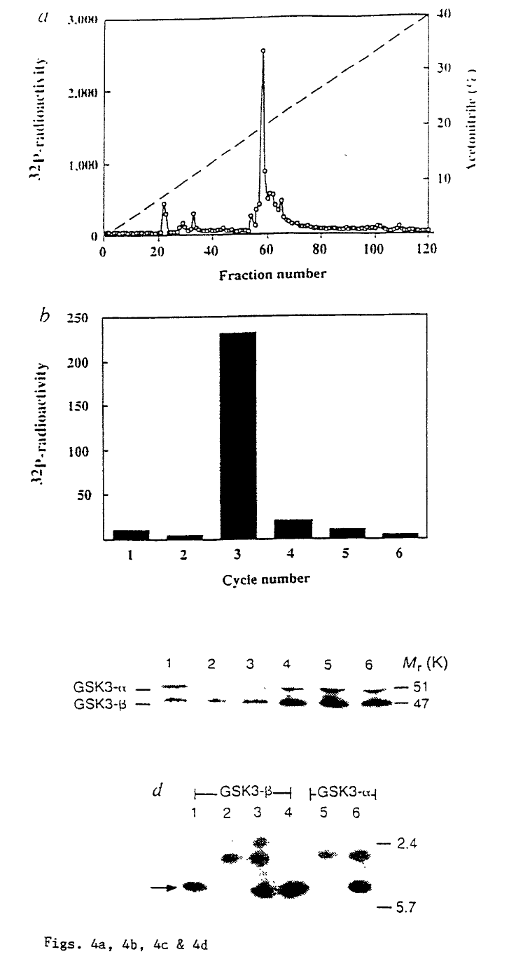

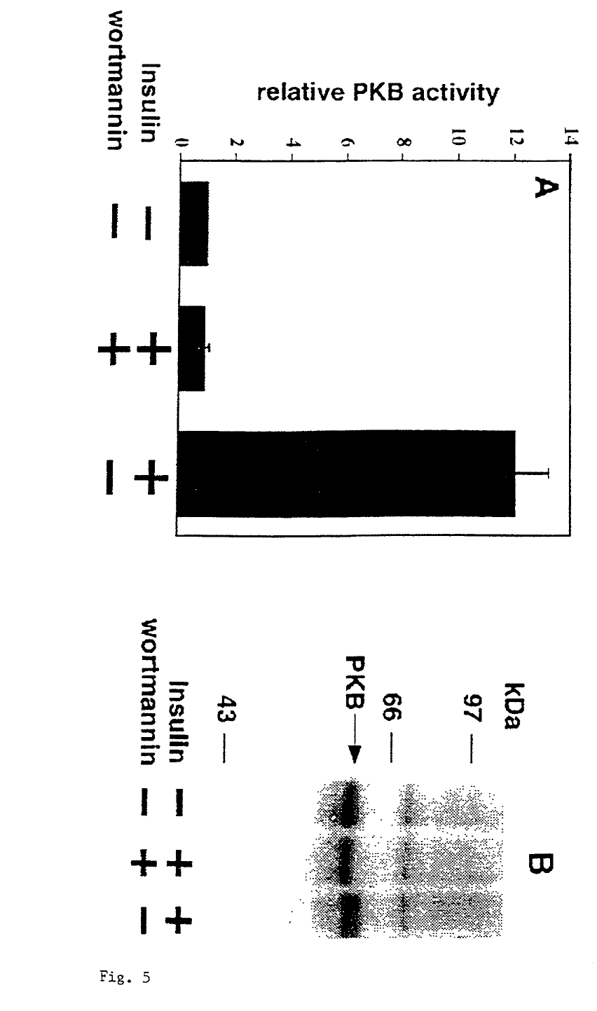

Insulin induces the activation and phosphorylation of PKBα in L6 myotubes. Three 10 cm dishes of L6 myotubes were ³²P-labelled and treated for 10 min with or without 100 nM wortmannin and then for 5 min with or without 100 nM insulin. PKBα was immunoprecipitated from the lysates and an aliquot (15%) assayed for PKBα activity (FIG. 5A). The activities are plotted ±SEM for 3 experiments relative to PKBα derived from unstimulated cells which was 10 mU/mg. The remaining 85% of the immunoprecipitated PKBα was alkylated with 4-vinylpyridine, electrophoresed on a 10% polyacrylamide gel (prepared without SDS to enhance the phosphorylation-induced decrease in mobility) and autoradiographed. The positions of the molecular mass markers glycogen phosphorylase (97 kDa), bovine serum albumin (66 kDa) and ovalbumin (43 kDa) are marked.

Under these conditions, insulin stimulation resulted in a 12-fold activation of PKBα (FIG. 5A) and was accompanied by a 1.9±0.3-fold increase in ³²P-labelling (4 experiments) and retardation of its mobility on SDS-polyacrylamide gels (FIG. 5B). The activation of PKBα, the increase in its ³²P-labelling and reduction in electrophoretic migration were all abolished by prior incubation of the cells with 100 nM wortmannin. Phosphoamino acid analysis of the whole protein revealed that ³²P-labelled PKBα was phosphorylated at both serine and threonine residues and that stimulation with insulin increased both the ³²P-labelling of both phosphoamino acids (data not shown).

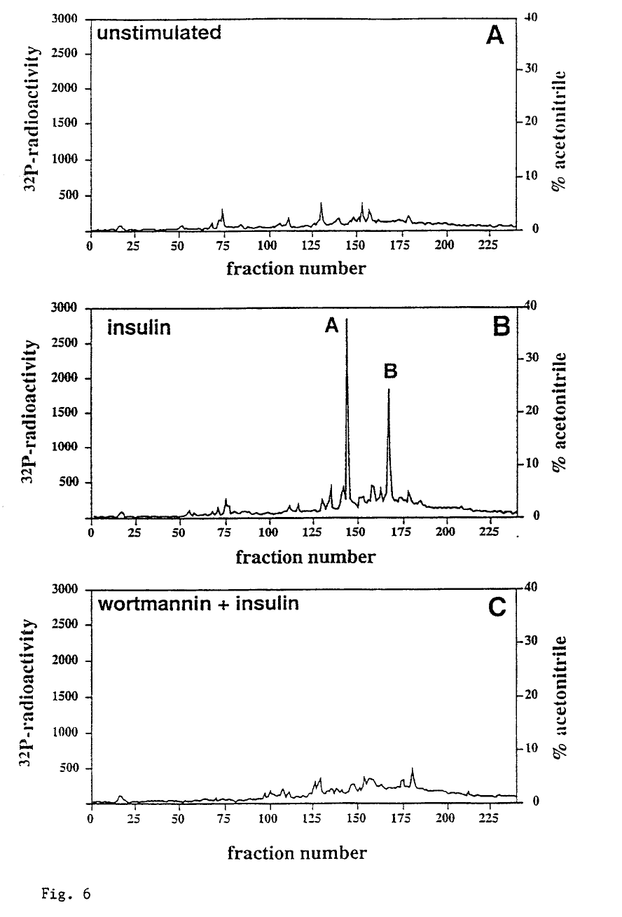

FIG. 6. Insulin stimulation of L6 myotubes induces the phosphorylation of two peptides in PKBα. Bands corresponding to ³²P-labelled PKBα from FIG. 5B were excised from the gel, treated with 4-vinylpyridine to alkylate cysteine residues, digested with trypsin and chromatographed on a Vydac 218TP54 C18 column (Separations Group, Hesperia, Calif.) equilibrated with 0.1% (by vol) trifluoroacetic acid (TFA), and the columns developed with a linear acetonitrile gradient (diagonal line). The flow rate was 0.8 ml/min and fractions of 0.4 ml were collected (A) tryptic peptide map of ³²P-labelled PKBα from unstimulated L6 myotubes; (B) tryptic peptide map of ³²P-labelled PKBα from insulin-stimulated L6 myotubes; (C) tryptic peptide map of ³²P-labelled PKBα from L6 myotubes treated with wortmannin prior to insulin. The two major ³²P-labelled peptides eluting at 23.7% and 28% acetonitrile are named Peptide A and Peptide B, respectively. Similar results were obtained in four (A and B) and two (C) experiments.

No major ³²P-labelled peptides were recovered from ³²P-labelled PKBα derived from unstimulated L6 myotubes (FIG. 6A) indicating that, in the absence of insulin, there was a low level phosphorylation at a number of sites. However, following stimulation with insulin, two major ³²P-labelled peptides were observed, termed A and B (FIG. 6B), whose ³²P-labelling was prevented if the myotubes were first preincubated with wortmannin (FIG. 6C).

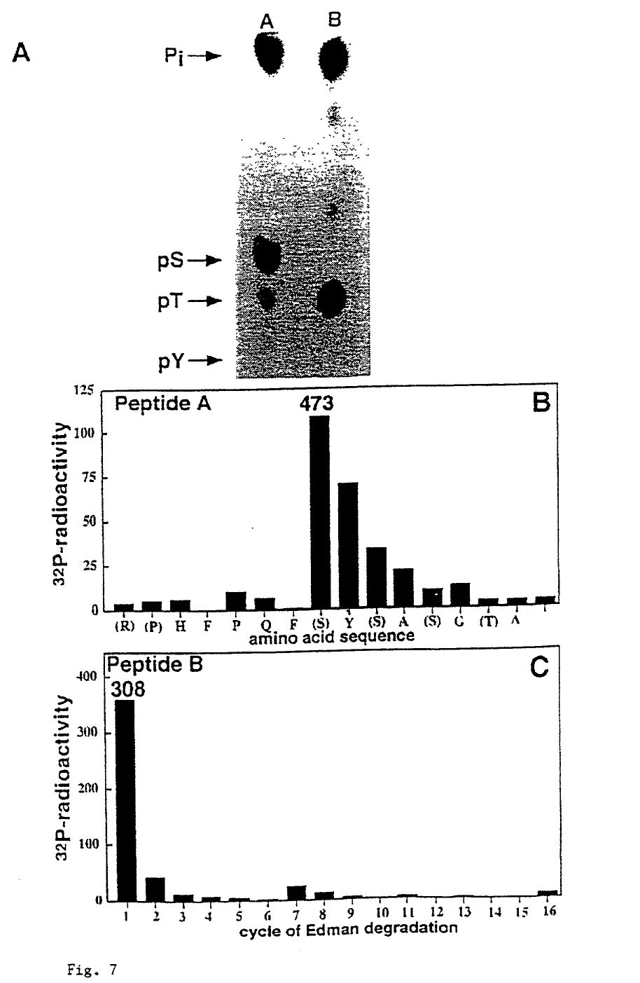

FIG. 7. Identification of the phosphorylation sites in peptides A and B. (A) Peptides A and B from FIG. 5B (1000 cpm) were incubated for 90 min at 110° C. in 6M HCl, electrophoresed on thin layer cellulose at pH 3.5 to resolve orthophosphate (Pi), phosphoserine (pS), phosphthreonine (pT) and phosphotyrosine (pY) and autoradiographed. (B) Peptide A (FIG. 5B) obtained from 50 10 cm dishes of ³²P-labelled L6 myotubes was further purified by chromatography on a microbore C18 column equilibrated in 10 mM ammonium acetate pH 6.5 instead of 0.1% TFA. A single peak of ³²P-radioactivity was observed at 21% acetonitrile which coincided with a peak of 214 nm absorbance. 80% of the sample (1 pmol) was analysed on an Applied Biosystems 476A sequencer to determine the amino acid sequence, and the phenylthiohydantoin (Pth) amino acids identified after each cycle of Edman degradation are shown using the single letter code for amino acids. The residues in parentheses were not present in sufficient amounts to be identified unambiguously. To identify the site(s) of phosphorylation, the remaining 20% of the sample (600 cpm) was then coupled covalently to a Sequelon arylamine membrane and analysed on an Applied Biosystems 470A sequencer using the modified programme described by Stokoe et al. (1992). 32P radioactivity was measured after each cycle of Edman degradation. (C) Peptide B from FIG. 2B (800 cpm) was subjected to solid phase sequencing as in (B).

Peptide A was phosphorylated predominantly on serine while peptide B was labelled on threonine (FIG. 7A). Amino acid sequencing established that peptide A commenced at residue 465. only a single burst of ³²P-radioactivity was observed after the eighth cycle of Edman degradation (FIG. 7B), demonstrating that insulin stimulation of L6 myotubes had triggered the phosphorylation of PKBα at Ser-473, which is located 9 residues from the C-terminus of the protein. Phosphopeptide B was only recovered in significant amounts if ³²P-labelled PKBα was treated with 4-vinylpyridine prior to digestion with trypsin, indicating that this peptide contained a cysteine residue(s), and a single burst of 32 p radioactivity was observed after the first cycle of Edman degradation (FIG. 7C). This suggested that the site of phosphorylation was residue 308, since it is the only threonine in PKBa that follows a lysine or arginine residue and which is located in a tryptic peptide containing a cysteine residue (at position 310). The acetonitrile concentration at which phosphopeptide B is eluted from the C18 column (28%) and its isoelectric point (4.0) are also consistent with its assignment as the peptide comprising residues 308-325 of PKBα. The poor recoveries of Peptide B during further purification at pH 6.5 prevented the determination of its amino acid sequence, but further experiments described below using transiently transfected 293 cells established that this peptide does correspond to residues 308-325 of PKBα.

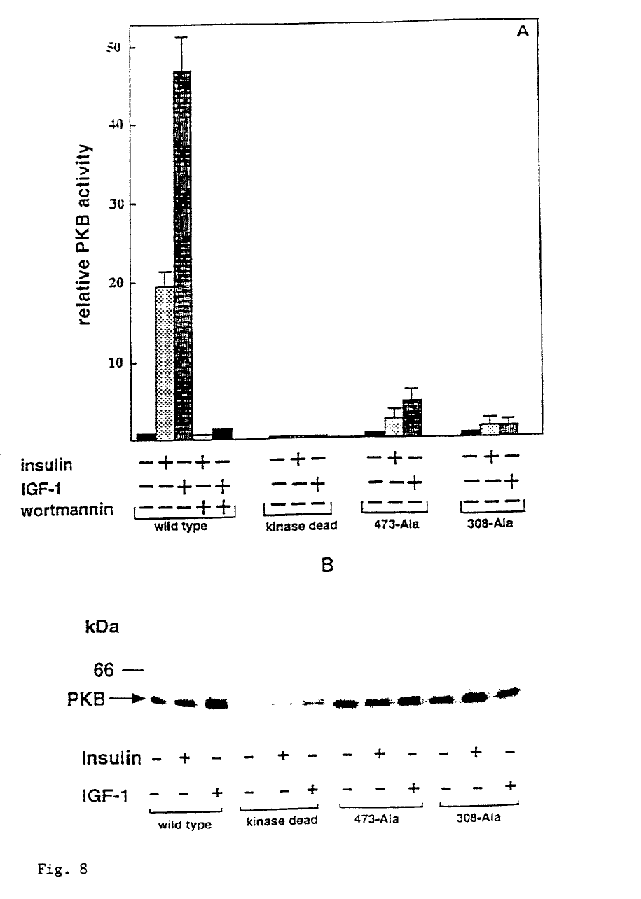

FIG. 8: Mapping the phosphorylation sites of PKBα in transiently transfected 293 cells. 293 cells were transiently transfected with DNA constructs expressing wild type PKBα, or a haemagglutonin epitope-tagged PKBα encoding the human protein, such as HA-KD PKBα, HA-473A PKBα or HA-308A PKBα. After treatment for 10 min with or without 100 nM wortmannin, the cells were stimulated for 10 min with or without 100 nM insulin or 50 ng/ml IGF-1 in the continued presence of wortmannin. PKBα was immunoprecipitated from the lysates and assayed, and activities corrected for the relative levels of expression of each HA-PKBα. The results are expressed relative to the specific activity of wild type HA-PKBα from unstimulated 293 cells (2.5±0.5 U/mg). (B) 20 μg of protein from each lysate was electrophoresed on a 10% SDS/polyacrylamide gel and immunoblotted using monoclonal HA-antibody. The molecular markers are those used in FIG. 5B.

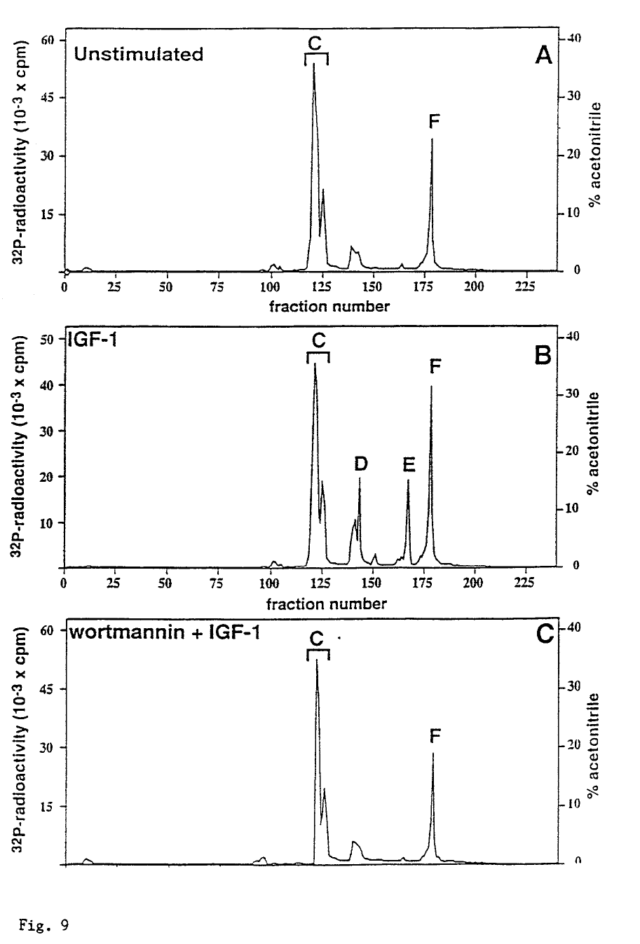

FIG. 9: IGF-1 stimulation of 293 cells induces the Phosphorylation of two peptides in transfected HA-PKBα. 293 cells transiently transfected with wild type HAPKBα DNA constructs were ³²P-labelled, treated for 10 min without (A, B) or with (C) 100 nM wortmannin and then for 10 min without (A) or with (B, C) 50 ng/ml IGF-1. The 32p labelled HA-PKBα was immunoprecipitated from the lysates, treated with 4-vinylpyridine, electrophoresed on a 10% polyacrylamide gel, excised from the gel and digested with trypsin. Subsequent chromatography on a C₁₈ column resolved four major phosphopeptides termed C, D, E and F. Similar results were obtained in 6 separate experiments for (A) and (B), and in two experiments for (C).

Stimulation with insulin and IGF-1 resulted in 20-fold and 46-fold activation of transfected PKBα, respectively (FIG. 8A), the half time for activation being 1 min, as found with other cells. Activation of PKBα by insulin or IGF-1 was prevented by prior incubation with wortmannin (FIG. 8A) and no activation occurred if 293 cells were transfected with vector alone and then stimulated with insulin or 1GF-1 (data not shown).

Two prominent ³²P-labelled peptides were present in unstimulated 293 cells (FIG. 9A). One, termed Peptide C, usually eluted as a doublet at 20-21% acetonitrile while the other, termed Peptide F, eluted at 29.7% acetonitrile. Stimulation with insulin or IGF-1 did not affect the ³²P-labelling of Peptides C and F (FIGS. 9A & B), but induced the ³²P-labelling of two new peptides, termed D (23.4% acetonitrile) and E (28% acetonitrile), which eluted at the same acetonitrile concentrations as peptides A and B from L6 myotubes (FIG. 6B) and had the same isoelectric points (7.2 and 4.0, respectively). Treatment of 293 cells expressing HA-PKBα with 100 nM wortmannin, prior to stimulation with IGF-1, prevented the phosphorylation of Peptides D and E, but had no effect on the 32p labelling of Peptides C and F (FIG. 9C).

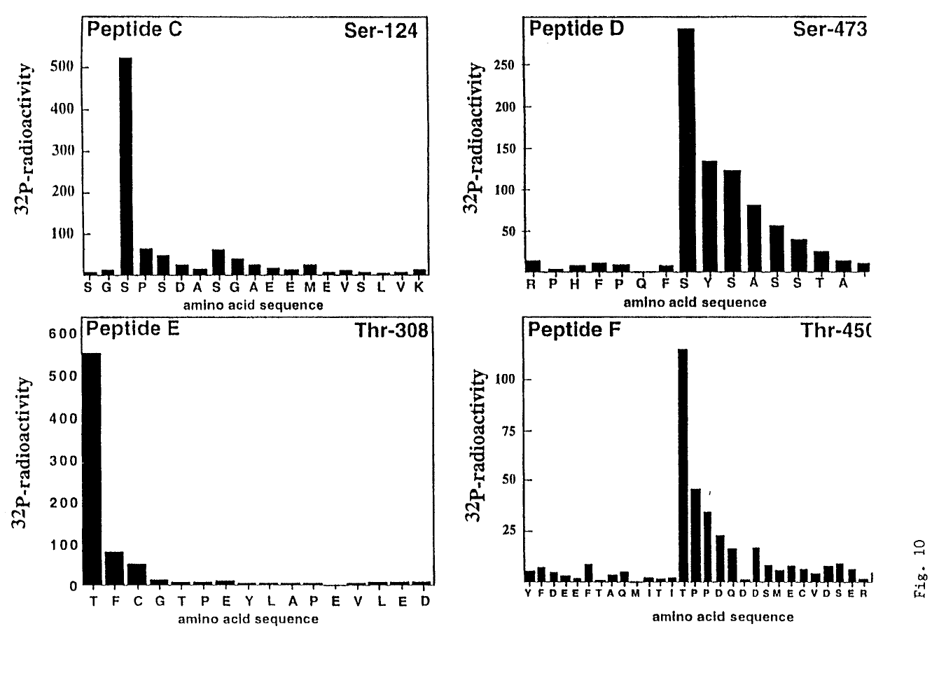

Peptides C, D, E and F were further purified by re-chromatography on the C18 column at pH 6.5 and sequenced. Peptides C gave rise to three separate (but closely eluting) ³²P-labelled peptides (data not shown). Amino acid sequencing revealed that all three commenced at residue 122 of PKBα and that Ser-124 was the site of phosphorylation (FIG. 10A). Peptide D only contained phosphoserine and, as expected, corresponded to the PKBα tryptic peptide commencing at residue 465 that was phosphorylated at Ser-473 (FIG. 10B). Peptide E, only contained phosphothreonine and amino acid sequencing demonstrated that it corresponded to residues 308-325, the phosphorylation site being Thr-308 (FIG. 10C). Peptide F only contained phosphothreonine and corresponded to the peptide commencing at residue 437 of PKBα phosphorylated at Thr-450 (FIG. 10D).

In the presence of phosphatidylserine, PKBα binds to PIP3 with submicromolar affinity (James et al., 1996, Frech et al.,1996). Phosphatidyl 4,5-bisphosphate and phosphatidyl 3,4 bisphosphate bind to PKBα with lower affinities and PI 3,5 bisphosphate and PI 3 phosphate did not bind at all under these conditions (James et al., 1996). The region of PKBα that interacts with PIP3 is almost certainly the PH domain, because the isolated PH domain binds PIP3 with similar affinity to PKBα itself (Frech et al., 1996) and because the PH domain of several other proteins, such as the PH-domains of, β-spectrin and phospholipase Cl, are known to interact specifically with other phosphoinositides (Hyvonen et al., 1995 and Lemmon et al., 1995).

The experiments described above were repeated using insulin instead of IGF-1. The results were identical, except that the ³²P-labelling of Peptides D and E was about 50% of the levels observed with IGF-1 (data not shown). This is consistent with the two-fold lower level of activation of PKBα by insulin compared with IGF-1 (FIG. 7A).

### EXAMPLE 3

MAPKAP Kinase-2 Phosphorylates Ser-473 of PKBα Causing Partial Activation

Ser-473 of PKBa lies in a consensus sequence Phe-x-x-Phe/Tyr-Ser/Thr-Phe/Tyr found to be conserved in a number of protein kinases that participate in signal transduction pathways (Pearson et al. 1995). In order to identify the Ser-473 kinase(s) we therefore chromatographed rabbit skeletal muscle extracts on CM-Sephadex, and assayed the fractions for protein kinases capable of phosphorylating a synthetic peptide corresponding to residues 465 to 478 of PKBα. These studies identified an enzyme eluting at 0.3 M NaCl which phosphorylated the peptide 465-478 at the residue equivalent to Ser-473 of PKBα. The Ser473 kinase co-eluted from CM-Sephadex with MAP kinase-activated protein (MAPKAP) kinase-2, (Stokoe et al, 1992) which is a component of a stress and cytokine-activated MAP kinase cascade (Rouse et al, 1994; Cuenda et al, 1995). The Ser-473 kinase continued to cofractionate with MAPKAPkinase-2 through phenyl-Sepharose, heparin-Sepharose, Mono S and Mono Q and was immunoprecipitated quantitatively by an anti-MAPKAP kinase-2 antibody (Gould et al, 1995) demonstrating that MAPKAP kinase-2 was indeed the Ser-473 kinase we had purified.

FIG. 11. HA-PKBα was immunoprecipitated from the lysates of unstimulated COS-1 cells expressing these constructs. (A) 0.5 μg of immunoprecipitated HA-PKBα was incubated with MAPKAP kinase-2 (50 U/ml), 10 mM magnesium acetate and 100 mM [γ³²P]ATP in a total of 40 μl of Buffer B. At various times, aliquots were removed and either assayed for PKBα activity (open circles) or for incorporation of phosphate into PKBα (closed circles). Before measuring PKBa activity, EDTA was added to a final concentration of 20 mM to stop the reaction, and the immunoprecipitates washed twice with 1.0 ml of buffer B containing 0.5 M NaCl, then twice with 1.0 ml of Buffer B to remove MAPKAP kinase-2. The results are presented as ±SEM for six determinations (two separate experiments) and PKBα activities are presented relative to control experiments in which HA-PKBα was incubated with MgATP in the absence of MAPKAP kinase-2 (which caused no activation). Phosphorylation was assessed by counting the ³²P-radioactivity associated with the band of PKBα after SDS/polyacrylamide gel electrophoresis. The open triangles show the activity of immunoprecipitated HA-KD PKBα phosphorylated by MAPKAP kinase-2. (B) HA-PKBα phosphorylated for 1 h with MAPKAP kinase-2 and 32P-γ-ATP as in (A) was digested with trypsin and chromatographed on a C18 column as described in the legend for FIG. 2. (C) The major ³²P-labelled peptide from (B) was analysed on the 470A sequencer as in FIG. 3 to identify the site of phosphorylation.

Bacterially expressed MAPKAP kinase-2 phosphorylated wild type HA-PKBα or the catalytically inactive mutant HA-PKBα in which Lys-179 had been mutated to Ala (data not shown) to a level approaching 1 mol per mole protein (FIG. 11A). Phosphorylation of wild-type PKBα was paralleled by a seven-fold increase in activity, whereas phosphorylation of the catalytically inactive mutant did not cause any activation (FIG. 11A). No phosphorylation or activation of wild type HA-PKBα occurred if MAPKAP kinase-2 or MgATP was omitted from the reaction (data not shown). Wild type HA-PKBα that had been maximally activated with MAPKAP kinase-2, was completely dephosphorylated and inactivated by treatment with protein phosphatase 2A (data not shown).

HA-PKBα that had been maximally phosphorylated with MAPKAP kinase-2 was digested with trypsin and C18 chromatography revealed a single major ³²P-labelled phosphoserine-containing peptide (FIG. 11B). This peptide eluted at the same acetonitrile concentration (FIG. 11B) and had the same isoelectric point of 7.2 (data not shown) as the 32 p labelled tryptic peptide containing Ser-473 (compare FIGS. 11B and 6B). Solid phase sequencing gave a burst of ³²P-radioactivity after the eighth cycle of Edman degradation (FIG. 11C), establishing that Ser-473 was the site of phosphorylation. The same ³²P-peptide was obtained following tryptic digestion of catalytically inactive HA-KD PKBα that had been phosphorylated with MAPKAP kinase-2 (data not shown).

### EXAMPLE 4

Phosphorylation of Thr-308 and Ser-473 Causes Synergistic Activation of PKBα

The experiments described above demonstrated that phosphorylation of Ser-473 activates PKBα in vitro but did not address the role of phosphorylation of Thr-308, or how phosphorylation of Thr-308 might influence the effect of Ser-473 phosphorylation on activity, or vice versa. We therefore prepared haemagglutonin (HA)-tagged PKBα DNA constructs in which either Ser-473 or Thr-308 would be changed either to Ala (to block the effect of phosphorylation) or to Asp (to try and mimic the effect of phosphorylation).

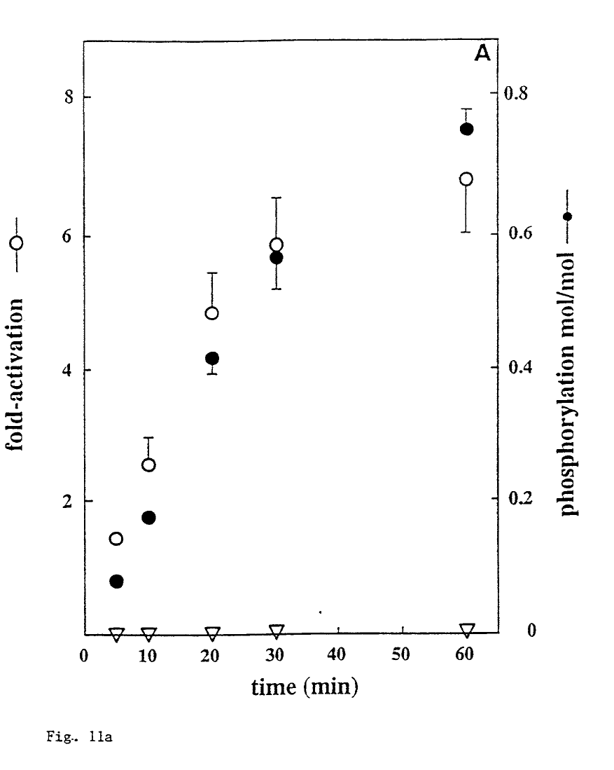

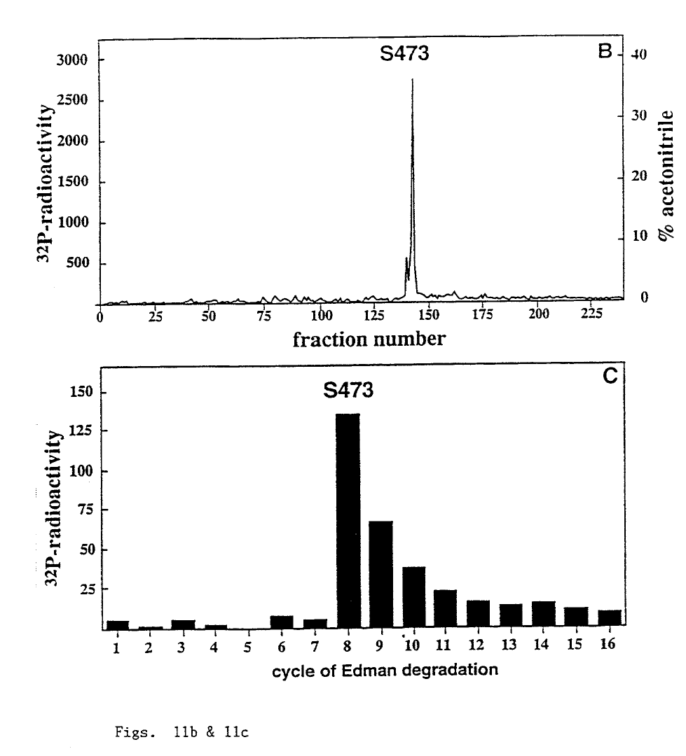

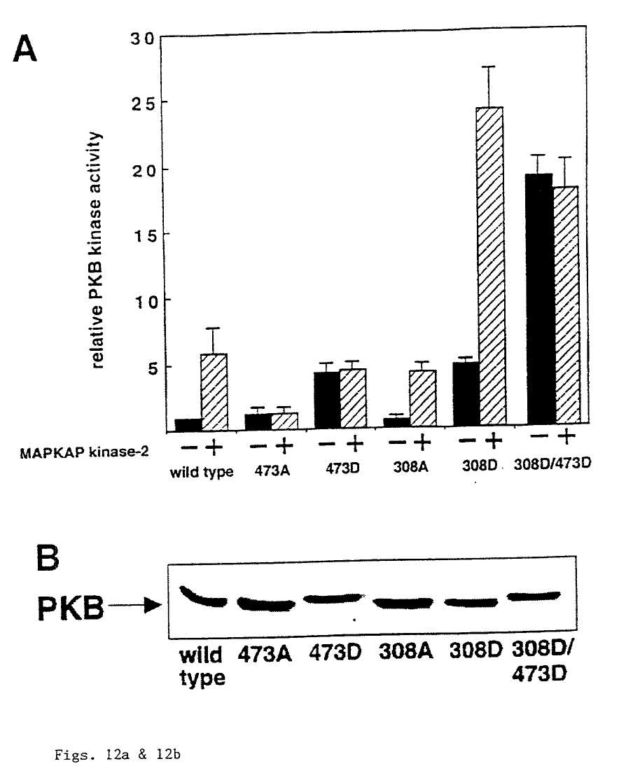

FIG. 12. Activation of HA-PKBα mutants in vitro by MAPKAP kinase-2. (A) Wild type and mutant HA-PKBα proteins were immunoprecipitated from the lysates of unstimulated COS-1 cells expressing these constructs and incubated for 60 min with MgATP in the absence (−, filled bars) or presence (+, hatched bars) of MAPKAP kinase-2 and MgATP (50 U/ml). The PKBα protein was expressed as similar levels in each construct and specific activities are presented relative to wild type HA-PKBα incubated in the absence of MAPKAP kinase-2 (0.03 U/mg). The re sults are shown as the average ±SEM for 3 experiments. (B) 20 μg of protein from each lysate was electrophoresed on a 10 % SDS/polyacrylamide gel and immunoblotted using monoclonal HA-antibody.

All the mutants were expressed at a similar level in serum-starved COS-1 cells (data not shown) and the effects of maximally phosphorylating each of them at Ser-473 is shown in FIG. 12A. Before phosphorylation with MAPKAP kinase-2 the activity of HA-473A PKBα was similar to that of unstimulated wild type HA-PKBα and, as expected, incubation with MAPKAP kinase-2 and MgATP did not result in any further activation of HA-473A PKBα. In contrast, the activity of HA-473D PKBα was five-fold to six-fold higher than that of unstimulated wild type HAPKBα protein, and similar to that of wild-type HA-PKBα phosphorylated at Ser-473. As expected, HA-473D PKBα was also not activated further by incubation with MAPKAP kinase-2 and MgATP. The activity of HA-308A PKBα was about 40% that of the unstimulated wild type enzyme, and after phosphorylation with MAPKAP kinase-2 is activity increased to a level similar to that of wild type HA-PKBα phosphorylated at Ser-473. Interestingly, HA-308D PKBα which (like HA-473D PK) was five-fold more active than dephosphorylated wild type HA-PKBα, was activated dramatically by phosphorylation of Ser-473. After incubation with MAPKAP kinase-2 and MgATP, the activity of HA-308D PKBα was nearly five-fold higher than that of wild type HA-PKBα phosphorylated at Ser-473 (FIG. 12B). These results suggested that the phosphorylation of either Thr-308 or Ser-473 leads to partial activation of PKBα in vitro, and that phosphorylation of both residues results in a synergistic activation of the enzyme. This idea was supported by further experiments in which both Thr-308 and Ser-473 were changed to Asp. When this double mutant was expressed in COS-1 cells it was found to possess an 18-fold higher specific activity than the dephosphorylated wild type protein. As expected, the activity of this mutant was not increased further by incubation with MAPKAP kinase-2 and MgATP (FIG. 12B).

### EXAMPLE 5

Phosphorylation of Both Thr-308 and Ser-473 is Required for a High Level of Activation of PKBα in vivo

FIG. 9. Effect of mutation of PKBα on its activation by insulin in 293 cells. (A) 293 cells were transiently transfected with DNA constructs expressing wild type PKBa, HA-D473-PKBα, and HA-308D/473D-PKBα. After treatment for 10 min with or without 100 nM wortmannin, cells were stimulated for 10 min with or without 100 nM insulin in the continued presence of wortmannin. PKBα was immunoprecipitated from the lysates and assayed, and activities corrected for the relative levels of HA-PKBα expression as described in the methods. The results are expressed relative to the specific activity of wild type HA-PKBα obtained from unstimulated 293 cells. (B) 20 μg of protein from each lysate was electrophoresed on a 10% SDS/polyacrylamide gel and immunoblotted using monoclonal HA-antibody.

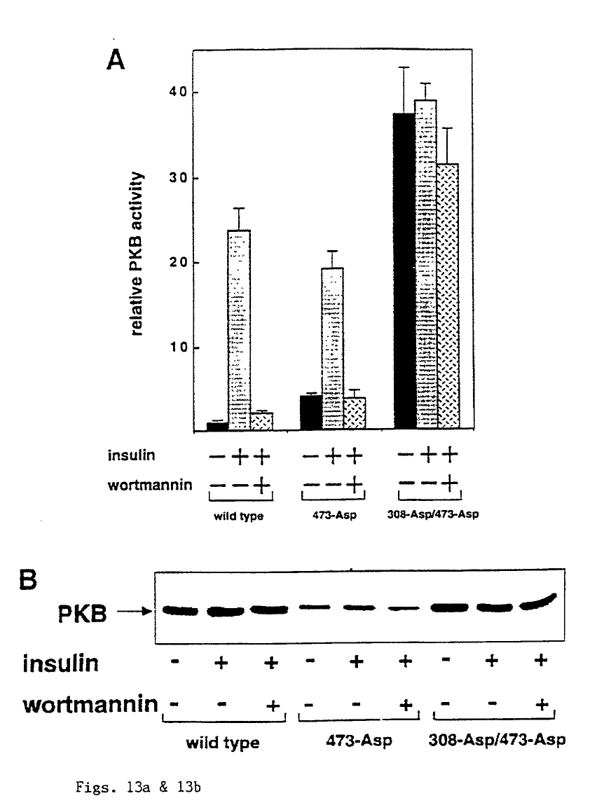

The basal level of activity of HA-473A PKBα derived from unstimulated cells was similar to that of wild type PKBα (FIG. 8A). Stimulation of 293 cells expressing HA-473A PKBα with insulin or IGF-1 increased the activity of this mutant three-fold and five-fold respectively; i.e. to 15% of the activity of wild type HA-PKBα which had been transiently expressed and stimulated under identical conditions. The basal activity of HA-308A PKBα in unstimulated cells was also similar to that of wild type HA-PKBα derived from unstimulated cells, but virtually no activation of this mutant occurred following stimulation of the cells with insulin or IGF-1. These data are consistent with in vitro experiments and indicate that maximal activation of PKBα requires phosphorylation of both Ser-473 and Thr-308 and that phosphorylation of both residues results in a synergistic activation of the enzyme. Consistent with these results, HA-473D PKBα displayed five-fold higher activity and the HA-308D/HA473D double mutant 40-fold higher activity than wild type HA-PKBα when expressed in unstimulated cells. Following stimulation with insulin, HA-473D PKBα was activated to a level similar to that observed with the wildtype enzyme, while the HA-308D/HA-473D double mutant could not be activated further (FIG. 13). As expected, activation of HA-473D PKBα by insulin was prevented by wortmannin, and the activity of the HA-308D/ HA-473D double mutant was resistant to wortmannin (FIG. 13).

### EXAMPLE 6

Phosphorylation of Thr-308 is not Dependent on Phosphorylation of Ser-473 or Vice Versa (in 293 Cells)

(FIG. 10) A 10 cm dish of 293 cells were transfected with either HA-308A PKBα or HA-473A PKBα, ³²P-labelled, then stimulated for 10 min with either IGF-1 (50 ng/ml) or buffer. The ³²P-labelled PKBα mutants were immunoprecipitated from the lysates, treated with 4-vinylpyridine, electrophoresed on a 10% polyacrylamide gel, excised from the gel and digested with trypsin, then chromatographed on a C18 column. The tryptic peptides containing the phosphorylated residues Ser-124, Thr-308, Thr-450, Ser-473 are marked and their assignments were confirmed by phosphoamino acid analysis and sequencing to identify the sites of phosphorylation (data not shown). The phosphopeptides containing Thr-308 and Ser-473 were absent if stimulation with IGF-1 was omitted, while the phosphopeptides containing Ser-124 and Thr-450 were present at similar levels as observed with wild-type PKBα (see FIG. 9A). Similar results were obtained in 3 separate experiments.

These experiments demonstrated that IGF-1 stimulation induced the phosphorylation of HA-473A PKBα at Thr-308, and the phosphorylation of HA-308A PKBα at Ser-473. Similar results were obtained after stimulation with insulin rather than IGF-I.

### EXAMPLE 7

IGF-1 or Insulin Induces Phosphorylation of Thr-308 and Ser-473 in a Catalytically Inactive Mutant of PKBα

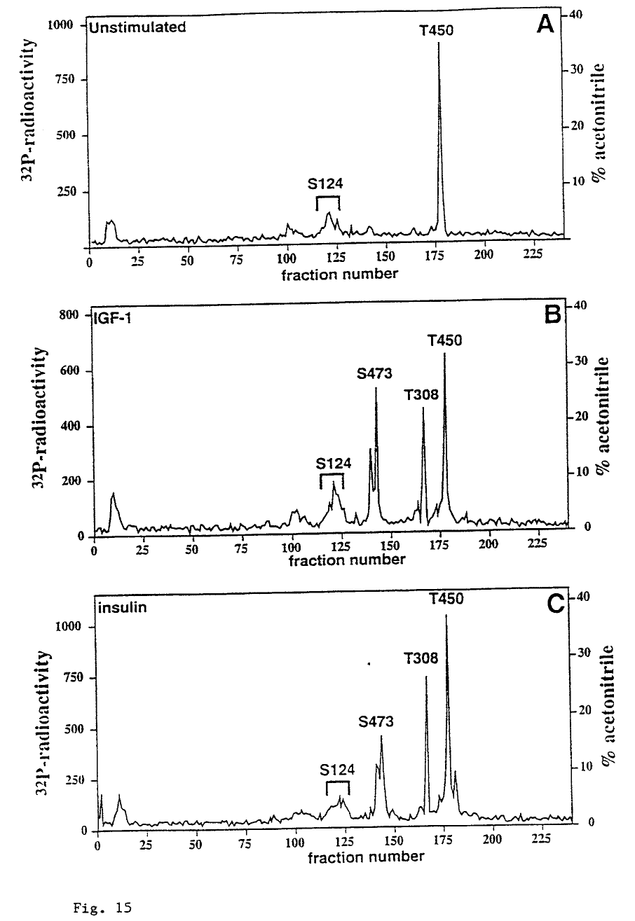

FIG. 15. The catalytically inactive PKBα mutant (HA-KD-PKBα) expressed in 293 cells is phosphorylated at Thr-308 and Ser-473 after stimulation with IGF-1. Each 10 cm dish of 293 cells transiently transfected with HA-KD-PKBα DNA constructs was ³²P-labelled and incubated for 10 min with buffer (A), 50 ng/ml IGF-1 (B) or 100 nM insulin (C). The ³²P-labelled HA-KD-PKBα was immunoprecipitated from the lysates, treated with 4 vinylpyridine, electrophoresed on a 10% polyacrylamide gel, excised from the gel and digested with trypsin, then chromatographed on a C18 column. The tryptic peptides containing the phosphorylated residues Ser-124, Thr-308, Thr-450 and Ser-473 are marked. Similar results were obtained in 3 separate experiments for (A) and (B), and in two experiments for (C).

This “kinase dead”mutant of PKBα, termed HA-KD-PKBα, in which Lys-179 was changed to Ala (see above) was transiently expressed in 293 cells and its level of expression found to be several-fold lower than that of wild type HA-PKBα expressed under identical conditions (FIG. 8B). As expected, no PKBαactivity was detected when 293 cells expressing HA-KD-PKBα, were stimulated with insulin or IGF-1 (FIG. 7A).

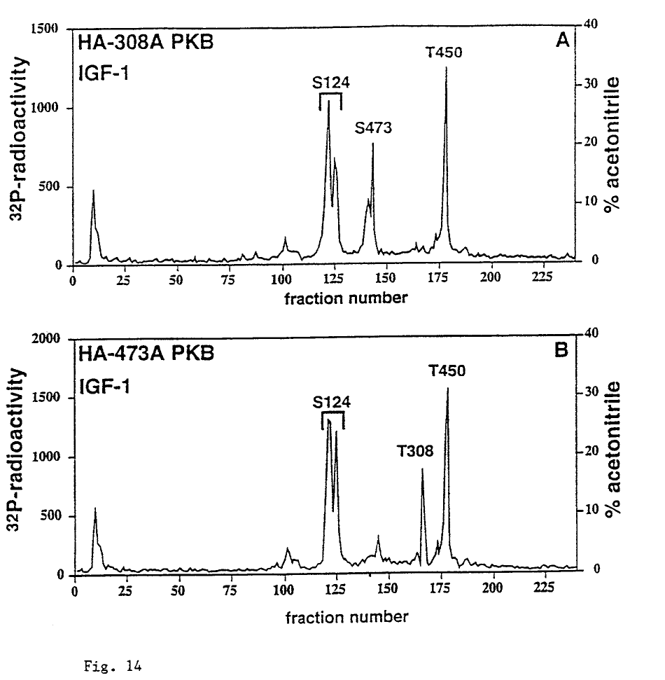

293 cells that had been transiently transfected with HA-KD-PKBα were ³²P-labelled, then stimulated with buffer, insulin or IGF-1. and sites on PKBα phosphorylated under these conditions were mapped. In contrast to wild type HA-PKBα from unstimulated 293 cells (FIG. 9), HA-KD PKBα was phosphorylated to a much lower level at Ser-124, but phosphorylated similarly at Thr-450 (FIG. 15A). Following stimulation with IGF-1 (FIG. 15B) or insulin (FIG. 14C) HA-KD-PKBα became phosphorylated at the peptides containing Thr-308 and Ser-473, the extent of phosphorylation of these sites being at least as high as wild type PKBα. Amino acid sequencing of these peptides established that they were phosphorylated at Thr-308 and Ser-473, respectively.

The above examples establish that PKB influences GSK3 activity; that Thr-308 and Ser-473 are the major residues in PKBα that become phosphorylated in response to insulin or IGF-1 (FIGS. 2 and 5) and that phosphorylation of both residues is required to generate a high level of PKBα activity. Thus mutation of either Thr-308 or Ser-473 to Ala greatly decreased the activation of transfected PKBα by insulin or IGF-1 in 293 cells (FIG. 8). Moreover, PKBα became partially active in vitro when either Thr-308 or Ser-473 were changed to Asp or when Ser-473 was phosphorylated by MAPKAP kinase-2 in vitro, and far more active when the D308 mutant of PKBα was phosphorylated by MAPKAP kinase-2 or when Thr-308 and Ser-473 were both mutated to Asp (FIG. 12). Moreover, the D308/D473 double mutant could not be activated further by stimulating cells with insulin (FIG. 13). These observations demonstrate that the phosphorylation of Thr-308 and Ser-473 act synergistically to generate a high level of PKBα activity.

Thr-308, and the amino acid sequence surrounding it, is conserved in rat PKBβ and PKBγ but, interestingly, Ser-473 (and the sequence surrounding it) is only conserved in PKBβ. In rat PKBγ, Ser-473 is missing because the C-terminal 23 residues are deleted. This suggests that the regulation of PKBγ may differ significantly from that of PKBα and PKBβ in the rat.

Thr-308 is located in subdomain VIII of the kinase catalytic domain, nine residues upstream of the conserved Ala-Pro-Glu motif, the same position as activating phosphorylation sites found in many other protein kinases. However, Ser-473 is located C-terminal to the catalytic domain in the consensus sequence Phe-Xaa-Xaa-Phe/Tyr-Ser/Thr-Phe/Tyr which is present in several protein kinases that participate in growth factor-stimulated kinase cascades, such as p70 S6 kinase, PKC and p90rsk (Pearson et al, 1995). However, it is unlikely that a common protein kinase phosphorylates this motif in every enzyme for the following reasons. Firstly, phosphorylation of the equivalent site in p70 S6 kinase is prevented by the immunosuppressant drug rapamycin (Pearson et al, 1995) which does not prevent the activation of PKBα by insulin (Cross et al, 1995) or is phosphorylation at Ser-473 (D. Alessi, unpublished work). Secondly, the equivalent residue in protein kinase C is phosphorylated constitutively and not triggered by stimulation with growth factors (Tsutakawa et al., 1995).

MAPKAP kinase-2 is a component of a protein kinase cascade which becomes activated when cells are stimulated with interleukin-1 or tumour necrosis factor or exposed cellular stresses (Rouse et al, 1994; Cuenda et al, 1995). MAPKAP kinase-2 phosphorylates PKBα stoichiometrically at Ser-473 (FIG. 11) and this finding was useful in establishing the role of Ser473 phosphorylation in regulating PKBα activity. However, although MAPKAP kinase-2 activity is stimulated to a small extent by insulin in L6 cells, no activation could be detected in 293 cells in response to insulin or IGF-1. Moreover, exposure of L6 cells or 293 cells to a chemical stress (0.5 mM sodium arsenite) strongly activated MAPKAP kinase-2 (D. Alessi, unpublished work) as found in other cells (Rouse et al, 1994; Cuenda et al, 1995), but did not activate PKBα at all. Furthermore, the drug SB 203580 which is a specific inhibitor of the protein kinase positioned immediately upstream of MAPKAP kinase-2 (Cuenda et al, 1995), prevented the activation of MAPKAP kinase-2 by arsenite but had no effect on the activation of PKBα by insulin or IGF-1. Finally, the activation of PKBα was prevented by wortmannin (FIGS. 6 and 9), but wortmannin had no effect on the activation of MAPKAP kinase-2 in L6 or 293 cells. It should also be noted that the sequence surrounding Ser-473 of PKBα (HFPQFSY) does not conform to the optimal consensus for phosphorylation by MAPKAP kinase-2 which requires Arg at position n-3 and a bulky hydrophobic residue at position n-5, (where n is the position of the phosphorylated residue). The Km for phosphorylation of the synthetic peptide comprising residues 465-478 is nearly 100-fold higher than the Km for the standard MAPKAP kinase-2 substrate peptide (data not shown). It is therefore unlikely that MAPKAP kinase-2 mediates the phosphorylation of Ser-473 in vivo.

The enzyme(s) which phosphorylates Thr-308 and Ser-473 in vivo does not appear to be PKBα itself. Thus incubation of the partially active AsP-308 mutant with MgATP did not result in the phosphorylation of Ser-473, phosphorylation of the latter residue only occurring when MAPKAP kinase-2 was added (FIG. 11A, FIG. 12). Similarly, Thr-308 did not become phosphorylated when either the partially active D473 mutant or the partially active Ser-473 phosphorylated form of PKBα were incubated with MgATP. PKBα when bound to lipid vesicles containing phosphatidylserine and PIP3 also fails to activate upon incubation with MgATP (Alessi et al, 1996) and after transfection into 293 cells, a “kinase dead” mutant of PKBα became phosphorylated on Thr-308 and Ser-473 in response to insulin or IGF-1 (FIG. 14). Furthermore, HA-PKBα from either unstimulated or insulin-stimulated 293 cells failed to phosphorylate the synthetic C-terminal peptide comprising amino acids 467-480.

In unstimulated L6 myotubes, the endogenous PKBα was phosphorylated at a low level at a number of sites (FIG. 6A), but in unstimulated 293 cells the transfected enzyme was heavily phosphorylated at Ser-124 and Thr-450 (FIG. 10). Ser-124 and Thr-450 are both followed by proline residues suggesting the involvement of “proline-directed” protein kinases. Although, the phosphorylation of Ser-124 was greatly decreased when “kinase dead” PKBα was transfected into 293 cells (FIG. 14), it would be surprising if Ser-124 is phosphorylated by PKBα itself because the presence of a C-terminal proline abolishes the phosphorylation of synthetic peptides by PKBα (D. Alessi, unpublished work). Since transfected PKBα is inactive in unstimulated 293 cells (FIG. 12), phosphorylation of Ser-124 and Thr-450 clearly does not activate PKBα directly. Ser-124 is located in the linker region between the PH domain and the catalytic domain of the mammalian PKBα isoforms but, unlike Thr-450, is not conserved in the Drosophila homologue (Andjelkovic et al, 1995).

While we do not wish to be bound by hypotheses, the results described suggest that agonists which activate PI 3-kinase are likely to stimulate PKBα activity via one of the following mechanisms. Firstly, PIP3 or PI3,4-bisP may activate one or more protein kinases which then phosphorylate PKBα at Thr-308 and Ser-473. Secondly, the formation of PIP3 may lead to the recruitment of PKBα to the plasma membrane where it is activated by a membrane-associated protein kinase(s). The membrane associated Thr-308 and Ser-473 kinases might themselves be activated by PIP3 and the possibility that Thr-308 and/or Ser-473 are phosphorylated directly by PI 3-kinase has also not been excluded, because this enzyme is known to phosphorylate itself (Dhand et al, 1994) and other proteins (Lam et al, 1994) on serine residues.

### EXAMPLE 8

Molecular Basis for Substrate Specificity of PKB

PKBα has been shown to influence GSK3 activity. GSK3α and GSK3β are phosphorylated at Ser-21 and Ser-9, respectively, by two other insulin-stimulated protein kinases, namely p70 S6 kinase and MAP kinase-activated protein kinase-1 (MAPKAP-K1, also known as p90 S6 kinase). However, these enzymes are not rate-limiting for the inhibition of GSK3 by insulin in L6 myotubes because specific inhibitors of their activation (rapamycin-p70 S6 kinase; PD 98059-MAPKAP kinase-1) have no effect (Cross et al., 1995).

The activation of PI 3-kinase is essential for many of the effects of insulin and growth factors, including the stimulation of glucose transport, fatty acid synthesis and DNA synthesis, protection of cells against apoptosis and actin cytoskeletal rearrangements (reviewed in Carpenter et al., 1996). These observations raise the question of whether PKBα mediates any of these events by phosphorylating other proteins. To address this issue we characterised the substrate specificity requirements of PKBα. We find that the optimal consensus sequence for phosphorylation by PKBα is the motif Arg-Xaa-Arg-Yaa-Zaa-Ser/Thr-Hyd, where Yaa and Zaa are small amino acids (other than glycine) and Hyd is a large hydrophobic residue (such as Phe or Leu). We also demonstrate that PKBα phosphorylates histone H2B (a substrate frequently used to assay PKBα in vitro) at Ser-36 which lies in an Arg-Xaa-Arg-Xaa-Xaa-Ser-Hyd motif. These studies identified a further PKBα substrate (Arg-Pro-Arg-Ala-Ala-Thr-Phe) that, unlike other peptides, is not phosphorylated to a significant extent by either p70 S6 kinase or MAPKAP-K1.

Results

### Preparation of Protein Kinase Bα

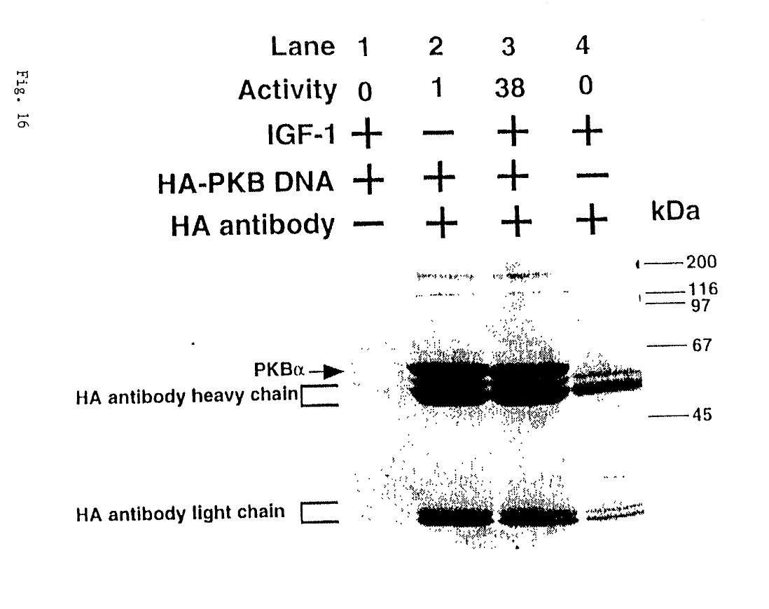

In order to examine the substrate specificity of PKBα, it was first necessary to obtain a kinase preparation that was not contaminated with any other protein kinase activities. 293 cells were therefore transiently transfected with a DNA construct expressing haemagglutonin-tagged PKBα (HA-PKBα), stimulated with IGF-1 and the HA-PKBα immunoprecipitated from the lysates). IGF-1 stimulation resulted in a 38-fold activation of PKBα (FIG. 16) and analysis of the immunoprecipitates by SDS-polyacrylamide gel electrophoresis revealed that the 60 kDa PKBα was the major protein staining with coomassie Blue apart from the heavy and light chains of the haemagglutonin monoclonal antibody (FIG. 16, Lanes 2 and 3). The minor contaminants were present in control immunoprecipitates derived from 293 cells transfected with an empty pCMV5 vector but lacked HA-PKB activity (FIG. 16, Lane 4). Furthermore, a catalytically inactive mutant HA-PKBα_immunoprecipitated from the lysates of IGF-1 stimulated 293 cells had no Crosstide kinase activity (Alessi et al., 1996). Thus, all the Crosstide activity in HA-PKB immunoprecipitates is catalysed by PKBα.

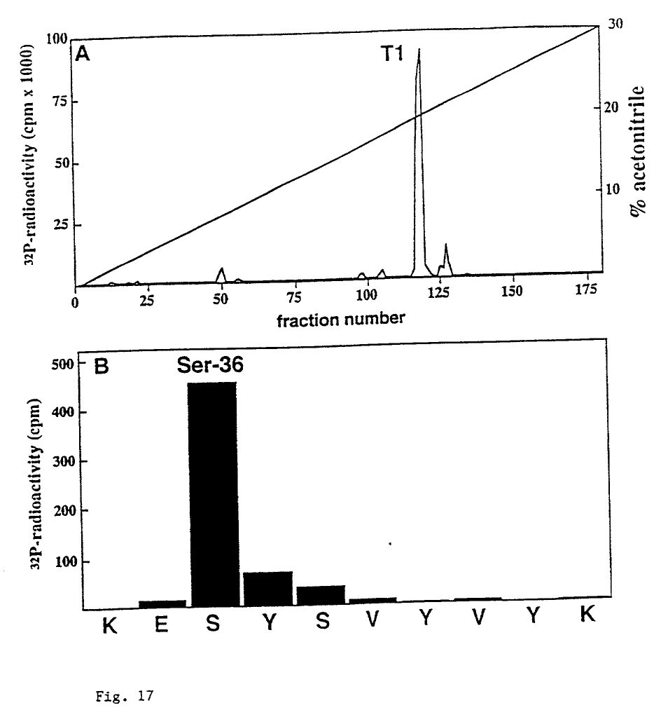

Identification of the residues in histone H2B Phosphorylated by PKBα. Currently, three substrates are used to assay PKBα activity in different laboratories, histone H2B, MBP and Crosstide. PKBα phosphorylated Crosstide with a Km of 4 μM and a Vmax of 260 U/mg (Table 7.1 A, peptide 1), histone H2B with a Km of 5 μM and a Vmax of 68 U/mg, and MBP with a Km of 5 μM and a Vmax of 25 U/mg. Thus the Vmax of histone H2B and MBP are 4-fold and 10-fold lower than for Crosstide. In order to identify the residue(s) in histone H2B phosphorylated by PKBα, ³²P-labelled histone H2B was digested with trypsin (see Methods) and the resulting peptides chromatographed on a C18 column at pH 1.9. Only one major ³²P-labelled peptide (termed T1) eluting at 19.5% acetonitrile was observed (FIG. 17A), The peptide contained phosphoserine (data not shown), its sequence commenced at residue 34 of histone H2B and a single burst of radioactivity occurred after the third cycle of Edman degradation (FIG. 17B), demonstrating that PKBα phosphorylates histone H2B at Ser-36 within the sequence Arg-Ser-Arg-Lys-Glu-Ser-Tyr. Thus, like the serine phosphorylated in Crosstide, Ser-36 of histone H2B lies in an Arg-Xaa-Arg-Xaa-Xaa-Ser-Hyd motif (where Hyd is a bulky hydrophobic residue -Phe in Crosstide, Tyr in H2B).

Molecular Basis for the Substrate Specificity of PKBα

To further characterise the substrate specificity requirements for PKBα, we first determined the minimum sequence phosphorylated efficiently by PKBα by removing residues sequently from the C-terminal and N-terminal end of Crosstide. Removal of the N-terminal glycine and up to three residues from the C-terminus had little effect on the kinetics of phosphorylation by PKBα (Table 7.1A, compare peptides 1 and 5). However any further truncation of either the N or C-terminus virtually abolished phosphorylation (Table 7.1A, peptides 8 and 9). The minimum peptide phosphorylated efficiently by PKBα (Arg-Pro-Arg-Thr-Ser-Ser-Phe) was found to be phosphorylated exclusively at the second serine residue as expected. Consistent with this finding, a peptide in which this serine was changed to alanine was not phosphorylated by PKBα (Table 7.1A, peptide 7). All further studies were therefore carried out using variants of peptide 5 in Table 7.1A (see below).

A peptide in which the second serine of peptide 5 (Table 7.1A) was replaced by threonine was phosphorylated with a Km of 30 μM and an unchanged Vmax (Table 7.1, peptide 6). All the ³²P-radioactivity incorporated was present as phosphothreonine and solid phase sequencing revealed that the peptide was only phosphorylated at the second threonine residue, as expected (data not shown). Thus PKBα is capable of phosphorylating threonine as well as serine residues, but has a preference for serine.

We next changed either of the two arginine residues in peptide 5 to lysine. These substitutions drastically decreased the rate of phosphorylation by PKBα (Table 7.1A, peptides 10 and 11) demonstrating a requirement for arginine (and not simply any basic residue) at both positions.

We then examined the effect of substituting the residues situated immediately C-terminal to the phosphorylated serine in peptide 5 (Table 7.1B). The data clearly demonstrate that the presence of a large hydrophobic residue at this position is critical for efficient phosphorylation, with the Km increasing progressively with decreasing hydrophobicity of the residue at this position (Table 7.1B, peptides 1 to 4). Replacement of the C-terminal residue with Lys increased the Km 18-fold and a substitution at this position with either Glu or Pro almost abolished phosphorylation (Table 7.1B, peptides 5-7).

Replacement of the Thr situated two residues N-terminal to the phosphorylated serine increased the Km with any amino acid tested (Table 7.1C). Substitution with Ala only increased Km by 2-3 fold, but substitution with other residues was more deleterious and with Asn (a residue of similar size and hydrophilicity to Thr) phosphorylation was almost abolished (Table 7.1C). Replacement of the Ser situated one residue N-terminal to the phosphorylated serine also increased the Km with any amino acid tested, but the effects were less severe than at position n-2 (Table 7.1C). When residues n-2 and n-1 were both changed to Ala, the resulting peptide RPRAASF was phosphorylated by PKBαwith a Km only 5-fold higher than RPRTSSF. In contrast the peptides RPRGGSF, RPRAGSF, and RPRGASF were phosphorylated less efficiently (Table 7.1C).

Comparison of the substrate specificity of PKBα with MAPKAP kinase-1, and p70 S6 kinase. Since MAPKAP-K1 and p70 S6 kinase phosphorylate the same residue in GSK3 phosphorylated by PKBα, and studies with synthetic peptides have established that MAPKAP-K1 and p70 S6 kinase also preferentially phosphorylate peptides in which basic residues are present at positions n-3 and n-5 (Leighton et al., 1995), we compared the specificities of MAPKAP-K1, p70 S6 kinase and PKBα in greater detail.

MAPKAP kinase-1 and p70 S6 kinase phosphorylate the peptides KKKNRTLSVA and KKRNRTLSVA with extremely low Km values of 0.2-3.3 μM, respectively (Table 7.2). However, these peptides were phosphorylated by PKBα with 50-900 fold higher Km values (Table 7.2A, peptides 1 and 2). The peptide KKRNRTLTV, which is a relatively specific substrate for p70 S6 kinase (Leighton et at., 1995) was also phosphorylated very poorly by PKBα (Table 7.2A, peptide 4).

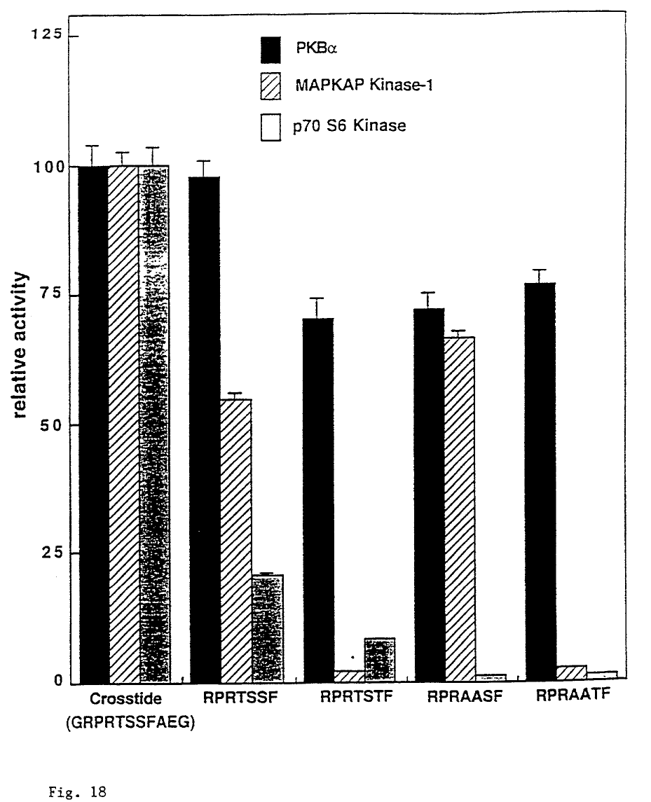

Crosstide is phosphorylated by p70 S6 kinase and MAPKAP kinase-1 with similar efficiency to PKBα ((Leighton et al., 1995); Table 7.2B-peptide 1 and FIG. 18). However, truncation of Crosstide to generate the peptide RPRTSSF was deleterious for phosphorylation by MAPKAP-K1 and even worse for p70 S6 kinase (Table 7.2B-peptides 1 and 2 and FIG. 18). Moreover, changing the phosphorylated serine in RPRTSSF to threonine increased the Km for phosphorylation by p70 S6 kinase much more than for PKBα and almost abolished phosphorylation by MAPKAP-K1 (Table 7.2B-peptide 3 and FIG. 18). The peptide RPRAASF was phosphorylated by MAPKAP-K1 with essentially identical kinetics to that of PKBα; however phosphorylation by p70 S6 kinase was virtually abolished (Table 7.2B-peptide 4 and FIG. 18). Based on these observations we synthesized the peptide RPRAATF. This peptide was phosphorylated by PKBα with a Km of 25 μM and similar Vmax to RPRTSSF, but was not phosphorylated to a significant extent by either MAPKAP-K1 or p70 S6 kinase (Table 7.2B-peptide 5, FIG. 18). In FIG. 18, the protein kinase concentration in the assays towards Crosstide were 0.2 U/ml, and each peptide substrate was assayed at a concentration of 30 μM. Filled bars denote PKBα activity, hatched bars MAPKAP kinase-1 activity, and grey bars p70 S6 kinase activity. The activities of each protein kinase are given relative to their activity towards Crosstide (100). The results are shown ±SEM for two experiments each carried out in triplicate.

Discussion

We have established that the minimum consensus sequence for efficient phosphorylation by PKBα is Arg-Xaa-Arg-Yaa-Zaa-Ser-Hyd, where Xaa is any amino acid, Yaa and Zaa are small amino acid other than glycine (Ser, Thr, Ala) and Hyd is a bulky hydrophobic residue (Phe, Leu) (Table 7.1). The heptapeptide with the lowest Km value was RPRTSSF, its Km of 5 μM being comparable to many of the best peptide substrates identified for other protein kinases. The Vmax for this peptide (250 nmoles min-1 mg-1) may be an underestimate because the PKBα was obtained by immunoprecipitation from extracts of IGF-1 stimulated 293 cells over-expressing this protein kinase, and a significant proportion of the PKBα may not have been activated by IGF-1 treatment.

The requirement for arginine residues at positions n-3 and n-5 (where n is the site of phosphorylation) seems important, because substituting either residue with lysine decreases phosphorylation drastically. Serine and threonine residues were preferred at positions n-1 and n-2, although the Km value was only increased about 5-fold if both of these residues were changed to Ala. Serine was preferred at position n, since changing it to threonine caused a six-fold increase in the Km. The peptide RPRAATF, which was phosphorylated with a Km of 25 μM and similar Vmax to RPRTSSF, may therefore be a better substrate for assaying PKBα in partially purified preparations, because unlike Crosstide, it contains only one phosphorylatable residue and is not phosphorylated significantly by MAPKAP-K1 or p70 S6 kinase (Table 7.2, FIG. 18 and see below).

The Proline at position n-4 was not altered in this study because it was already clear that this residue was not critical for the specificity of PKBα. Residue n-4 is proline in GSK3β but alanine in GSK3α. Both GSK3 isoforms are equally good substrates for PKBα in vitro (Cross et al., 1995), and the peptide GRARTSSFA (corresponding to the sequence in GSK3α) is phosphorylated by PKBα with a Km of 10 μM and Vmax of 230 U/mg (Table 7.1A, peptide 2). Moreover, in histone H2B, the residue located four amino acids N-terminal to the PKBα phosphorylation site is serine (FIG. 17). The presence of Glu and Lys at positions n-1 and n-2 may explain why histone H2B is phosphorylated by PKBα with a four-fold lower Vmax than the peptide RPRTSSF.

Two other protein kinases which are activated by insulin and other growth factors, p70 S6 kinase and MAPKAP-K1, require basic residues at positions n-3 and n-5 (Leighton et al., 1995), explaining why they also phosphorylate and inactivate GSK3 in vitro (Sutherland et al., 1993). Indeed, there is evidence that MAPKAP-K1 plays a role in the inhibition of GSK3 by EGF because, unlike inhibition by insulin which is prevented by inhibitors of PI 3-kinase, the inhibition of GSK3 by EGF is only suppressed partially by inhibitors of PI 3-kinase. Moreover, in NIH 3T3 cells, the inhibition of GSK3α and GSK3β by EGF is largely prevented by expression of a dominant negative mutant of MAP kinase kinase-1 (Eldar et al., 1995). In contrast, p70 S6 kinase is not rate limiting for the inhibition of GSK3 in the cells that have been examined so far because rapamycin, which prevents the activation of p70 S6 kinase by EGF or insulin, has no effect on the inhibition of GSK3 by these agonists (Cross et al., 1995 and Saito et al., 1994).

Additional similarities between p70 S6 kinase, MAPKAP-K1 and PKBα include the failure to phosphorylate peptides containing Pro at position n+1 and dislike of a lysine at the same position. This suggests that, in vivo, these kinases are unlikely to phosphorylate the same residues as MAP kinases (which phosphorylates Ser/Thr-Pro motifs) or protein kinase C (which prefers basic residues C-terminal to the site of phosphorylation). However, the present work has also revealed significant differences in the specificities of these enzymes. In particular MAPKAP-K1 and (to a lesser extent) p70 S6 kinase can tolerate substitution of the Arg at position n-5 by lysine whereas PKBα cannot (see Table 7.1A, Table 7.2A and (Leighton et al., 1995)). MAPKAP-K1 and p70 S6 kinase can also tolerate, to some extent, substitution of Arg at position n-3 by Lys. For example, the peptide KKRNKTLSVA is phosphorylated by MAPKAP-K1 and p70 S6 kinase with Km values of 17 and 34 μM, respectively, as compared to Km values of 0.7 and 1.5 μM for the peptide KKRNRTLSVA (Table 7.2A). In contrast, PKBα does not phosphorylate the peptide KKRNKTLSVA (Table 7.2A) or any other peptide that lacks Arg at position n-3. PKBα and p70 S6 kinase, but not MAPKAP-K1, phosphorylate Thr as well as Ser (Table 7.1A) and can phosphorylate peptides lacking any residue at position n+2 ((Leighton et al., 1995) and Table 7.2A), while PKBα and NAPKAP-K1, but not p70 S6 kinase, can tolerate substitution of both the n-1 and n-2 positions of the peptide RPRTSSF with Ala (Table 7.2B). These differences explain why the peptide RPRAATF is a relatively specific substrate for PKBα.

One of the best peptide substrates for MAPKAP-K1 and p70 S6 kinase (KKRNRTLSVA) was a poor substrate for PKBα (Table 7.2, peptide 2), despite the presence of Arg at positions n-3 and n-5. The presence of Leu at position n-1 and Val at position n+1 are likely to explain the high Km for phosphorylation, because PKBα prefers a small hydrophilic residue at the former position and a larger hydrophobic residue at the latter position (Tables 7.1 and 7.2).

### EXAMPLE 9

This example demonstrates that coexpression of GSK3 in 293 cells with either the wild type or a constitutively activated PKB results in GSK3 becoming phosphorylated and inactivated. However coexpression of a mutant of GSK3 in which Ser-9 is mutated to an Ala residue is not inactivated under these conditions. These experiments provide further evidence that PKBa activation can mediate the phosphorylation and inactivation of GSK3 in a cellular environment, and could be used as an assay system to search for specific PKB inhibitors.

Monoclonal antibodies recognising the sequence EFMPME (EE) antibodies and the (EQKLISEEDL) c-Myc purchased from Boehringer (Lewis, UK).

Construction of expression vectors and transfections into 293 cells. HA-PKBa, HA-KD-PKB and 308D/473D HA-PKBa was described previously (Alessi et al. 1996).

A DNA construct expressing human GSK3B with the EFMPME (EE) epitope tag at the N-terminus was prepared as follows: A standard PCR reaction was carried out using as a template the human GSK3β cDNA clone in the pBluescript SK+vector and the oligonucleotides GCGGAGATCTGCCACCATGGAGTTCATGCCCATGGAGTCAGGGCGGCCCAGAACC and GCGGTCCGGAACATAGTCCAGCACCAG that incorporate a bgl II site (underlined) and a Bspe I site (double underlined). A three-way ligation was then set up in which the resulting PCR product was subcloned as a Bgl II-Bspe I fragment together with the C-terminal Bspe I-Cla I fragment of GSK3β into the Bgl II-Cla I sites of the pCMV5 vector (Anderson et al., 1989). The construct was verified by DNA sequencing and purified using the Quiagen plasmid Mega kit according to the manufacturers protocol. The c-Myc GSK3, BA9 construct encodes GSK3β in which Ser-9 is mutated to Ala and possesses a c-myc epitope tag at the C-terminus and was prepared as described in Sperber et al., 1995. The c-Myc GSK3β A9 gene was then subcloned into xba I/ECOR I sites of the pCMV5 eukaryotic expression vector.

Cotransfection of GSK3β with PKBa and its assay. 293 cells growing on 10 cm diameter dishes were transfected with 10 ug of DNA constructs expressing EE-GSK3, Myc-GSK3A9 in the presence or absence of HA-PKB, HA-KD-PKB or HA-308D/473D-PKB exactly as described in Alessi et al., 1996. The cells were grown in the absence of serum for 16 h prior to lysis, and then lysed in 1.0 ml of ice-cold Buffer A (50 mM Tris/HCI pH 7.5, 1 mM EDTA 1 mM EGTA, 1% (by vol) Triton X100, 1 mM sodium orthopervanadate, 10 mM sodium glycerophosphate, 50 mM NaF, 5 mM sodium pyrophosphate, 1 uM Microcystin-LR, 0.27 M sucrose, 1 mM benzamidine, 0.2 mM phenylmethylsulphonyl fluoride, 10 ug/ml leupeptin, and 0.1% (by vol) 2-mercaptoethanol). The lysate was centrifuged at 4° C. for 10 min at 13, 000×g and an aliquot of the supernatant (100 ug protein) was incubated for 30 min on a shaking platform with 5 ul of protein G-Sepharose coupled to lug of EE monoclonal antibody. The suspension was centrifuged for 1 min at 13,000×g, the Protein G-Sepharose-antibody-EE-GSK3β complex washed twice with 1.0 ml of Buffer A containing 0.5 M NaCl, and three times with Buffer B (50 mM Tris pH 7.5, 0.1 mM EGTA, 0.01% (by vol) Brij-35 and 0.1% (by vol) 2-mercaptoethanol), and the immunoprecipitate assayed for GSK3 activity after incubation with either PP2A or microcystin inactivated PP2A as described previously (Cross et al., 1994).

Results

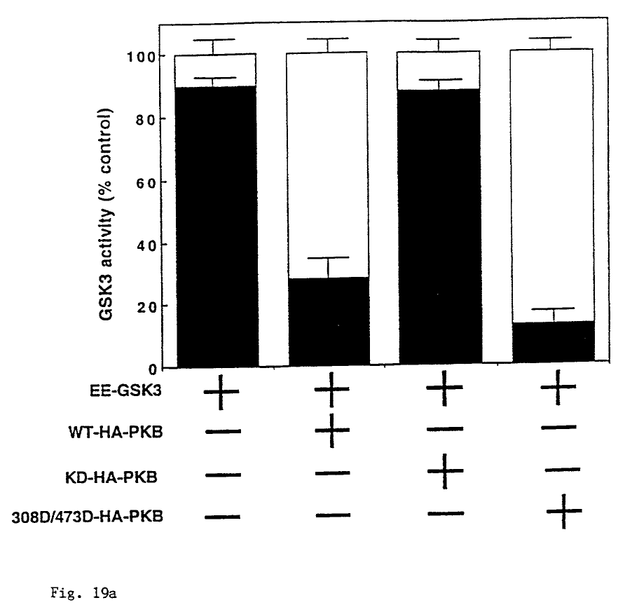

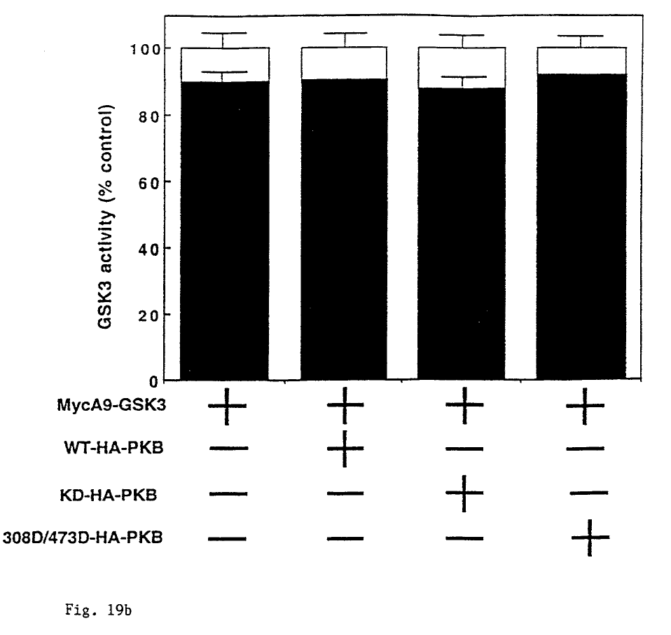

Cotransfection of GSK3β with PKBa in 293 cells results in GSK3 phosphorylation and inactivation Human embryonic kidney 293 cells were transfected with a DNA construct expressing EE-epitope tagged GSK3β either in the presence or absence of DNA constructs expressing wild type-PKBa, a catalytically inactive PKBa or a constitutively active HA-(308D/473D)-PKBa. Cells were serum starved for 16 h. 36 h post transfection the cells were lysed, and the GSK3β immunoprecipitated from the lysates using monoclonal EE antibodies and the GSK3β activity measured before and after treatment with PP2A. When EEGSK3β was expressed alone or in the presence of a catalytically inactive PKBa, treatment of the EE-GSK3β with PP2A only resulted in about a 12% increase in activity (FIG. 19A). However when EE-GSK3β was coexpressed with either the wild type PKBa or the constitutively activated 308D/473D-HA-PKBa, treatment of the EE-GSK3 from these cell lysates with PP2A resulted in a 68% and 85% increase in the GSK3 activity, respectively. Coexpression of Myc-GSK3β A9 with HA-PKB or the constitutively active 308D/473D-HA-PKBa did not result in any significant inactivation of this mutant of GSK3 as judged by its ability to be reactivated by PP2A (FIG. 19B). These data demonstrate that even in a cellular environment, PKBa is capable of phosphorylating GSK3β at Ser-9 and inactivation of the enzyme. To estimate the relative levels of EE-GSK3β and PKBa, EE-GSK3 and HA-PKBa were immunoprecipitated from equal volumes of cell lysate, and the immunoprecipitates run on an SDS-polyacrylamide gel, and the gel stained with Coomassie Blue. These experiments revealed that both the wild type HA-PKBa and the 308D/473D-PKBa were expressed at a 20 to 30-fold higher level than GSK3a, whereas KD-PKBa is expressed at a level that is about 5-fold lower than that of the wild type PKBa. Under the conditions used for the immunoprecipitations, no PKBa was co-immnuoprecipitated with GSK3β, or no GSK3β was co-immunoprecipitated with the PKBa (data not shown). Coexpression of EE-GSK3β with all forms of PKBa resulted in about a 2-3 fold decrease in the level of expression on EE-GSK3β compared to when it is expressed alone in cells.

### EXAMPLE 10

Basic Assay for Identifying Agents which Affect the Activity of PKB

A 40 μl assay mix was prepared containing protein kinase (0.2 U/ml) in 50 mM Tris/HCI pH 7.5, 0.1 mM EGTA, 0.1% (by vol) 2-mercaptoethanol, 2.5 μM PKI, protein kinase substrate (30 μM), and the indicated concentration of Ro-318220 or GC 109203X (test inhibitors). After incubation on ice for 10 min the reaction was started by the addition of 10 μl of 50 mM magnesium acetate and 0.5 mM [γ³²P]ATP (100-200 cpm/pmol). For the assay of mixed isoforms of PKC 20 μM diacylglycerol, 0.5 mM CaCl₂, and 100 μM phosphatidylserine were also present in the incubations. The assays were carried out for 15 min at 30° C., then terminated and analysed as described (Alessi 1995). One unit of activity was that amount of enzyme that catalysed the phosphorylation of 1 nmol os substrate in 1 min. The final concentration of DMSO in each assay was 1% (by vol). This concentration of DMSO does not inhibit any of these enzymes. Mixed isoforms of PKC were assayed using histone H1 as substrate, while MAPKAP-K1β and p70 S6 kinase were assayed using the peptide KKRNRTLSVA (Leighton 1995). Protein kinase B was assayed with the peptide GRPRTSSFAEG [9] and MAPKAP-K2 was assayed with the peptide KKLNRTLSVA (Stokoe 1993). p42 MAP kinase was assayed using MBP, and MAPKK-1, and c-Raf1 were assayed as described in Alessi 1995.

Results

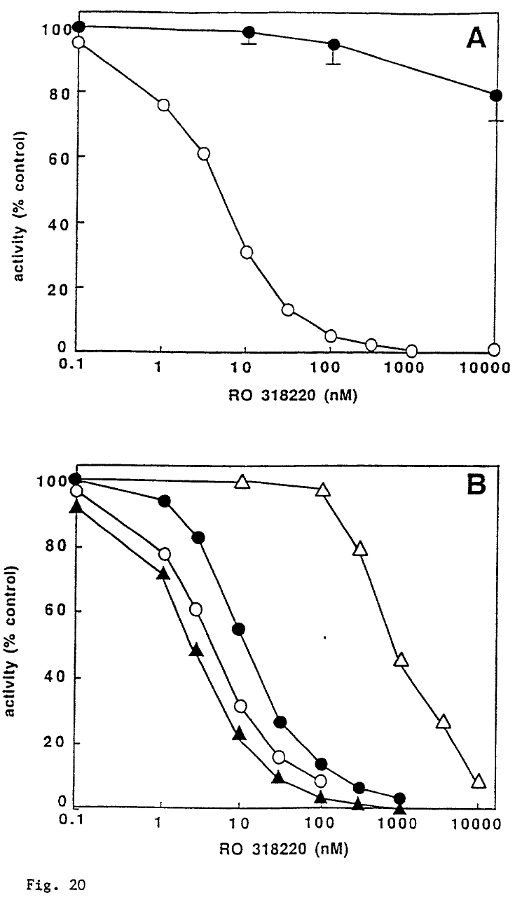

Effect of Ro 318220 and GF 109203X on protein kinases activated by growth factors, cytokines and cellular stresses. The mixed isoforms of PKC were potently inhibited by Ro 318220, with an IC₅₀ of 5 nM in our assay (FIG. 20A). In contrast, a number of protein kinases activated by growth factors (c-Raf1, MAPKK-1, p42 MAP kinase) and one protein kinase that is activated by cellular stresses and proinflammatory cytokines (MAPKAP-K2) were not inhibited significantly by Ro 318022 in vitro (FIG. 20A). Protein kinase B, an enzyme that is activated in response to insulin and growth factors was inhibited by Ro 318220 (IC₅₀ of 1 μM, FIG. 20B) similar to the IC₅₀ for PKA. However, to our surprise, MAPKAP-K1B an enzyme which lies immediately downstream of p42 and p44 MAP kinases and which is activated in response to every agonist that stimulates this pathway, was inhibited by Ro 318220 even more potently than the mixed PKC isoforms (IC₅₀=3 nm, FIG. 20B). The p70 S6 kinase, which lies on a distinct growth factor-stimulated signalling pathway from MAPKAP-K1B, was also potently inhibited by Ro 318220 (IC₅₀=15 nM, FIG. 20B).

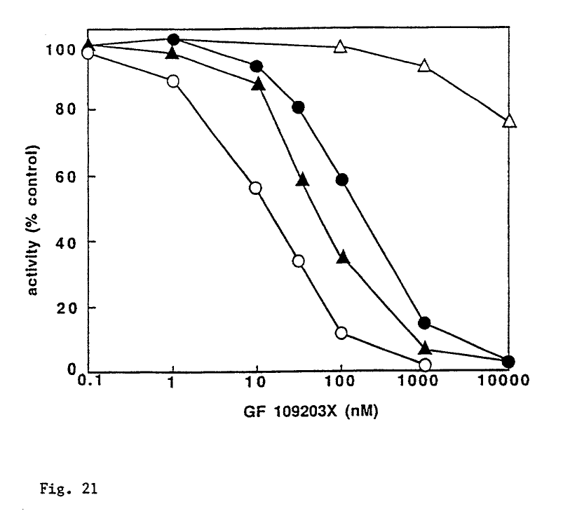

Similar results were obtained using GF 109203X instead of Ro 3318220. As reported previously (Toullec et al., 1991), GC 109203X inhibited the mixed isoforms of PKC (IC₅₀=30 nM) without inhibiting protein kinase B (FIG. 21) or c-Raf, MAPKK-1 and p42 MAP kinase (data not shown). However MAPKAP-K1B and p70 S6 kinase were potently inhibited by this compound with IC₅₀ values of 50 nM and 100 nM, respectively (FIG. 21).

General Materials and Methods Tissue culture reagents, myelin basic protein (MBP), microcystin-LR, and IGF-1 were obtained from Life Technologies Inc. (Paisley, UK), insulin from Novo-Nordisk (Bagsvaerd, Denmark), phosphate free Dulbecco's minimal essential medium (DMEM) from (ICN, Oxon, UK), Protein G-Sepharose and CH-Sepharose from Pharmacia (Milton Keynes, UK), alkylated trypsin from Promega (Southampton, UK), 4-vinylpyridine, wortmannin and fluroisothiocyanante-labelled antimouse IgG from goat from Sigma-Aldrich (Poole, Dorset, UK). Polyclonal antibodies were raised in sheep against the peptides RPHFPQFSYSASGTA (corresponding to the last 15 residues of rodent PKBα) and MTSALATMRVDYEQIK (corresponding to residues 352 to 367 of human MAPKAPkinase-2) and affinity purified on peptide-CH-Sepharose. Monoclonal HA antibodies were purified from the tissue culture medium of 12CA5 hybridoma and purified by chromatography on Protein GSepharose. The peptide RPRHFPQFSYSAS, corresponding to residues 465-478 of PKBα, was synthesized on an Applied Biosystems 430A peptide synthesizer. cDNA encoding residues 46-400 of human MAPKAP kinase-2 was expressed in E.coli as a glutathione S-transferase fusion protein and activated with p38/RK MAP KINASE by Mr A. Clifton (University of Dundee) as described previously (Ben-Levy et al., 1995).

Monoclonal antibodies recognising the haemagglutonin (HA) epitope sequence YPYDVPDYA, Protein G-Sepharose and histone H2B were obtained from Boehringer (Lewes, UK). MAPKAP kinase-1 (Sutherland et al., 1993) and p70 S6 kinases (Leighton et al., 1995) were purified from rabbit skeletal muscle and rat liver respectively.

Construction of expression vectors. The pECE constructs encoding the human HAPKBα and kinase-dead (K179A) HA-KD-PKBα have already been described (Andjelkovic at al., 1996). The mutants at Ser-473 (HA-473A PKBα and HA-473D PKBα were created by PCR using a 5′ oligonucleotide encoding amino acids 406-414 and mutating 3′ oligonucleotide encoding amino acids 468-480, and the resulting PCR products subcloned as CelII-EcoRI fragment into pECE.HA-PKBα. The Thr-308 mutants (HA-308A PKBα and HA308D PKBα) were created by the two-stage PCR technique (No et al., 1989) and subcloned as NotI-EcoRI fragments into pECE.HA-PKB. The double mutant HA-308D/473D PKB was made by subcloning the CelII-EcoRI fragment encoding 473D into pECE.HA-308D PKBα. For construction of cytomegalovirus-driven expression constructs, BglII-XbaI fragments from the appropriate pECE constructs were subcloned into the same restriction sites of the pCMVS vector (Andersson et al., 1989).

All constructs were confirmed by restriction analysis and sequencing and purified using Quiagen Plasmid Maxi Kit according to the manufacturer's protocol. All oligonucleotide sequences are available upon request.

³²P-labelling of L6 myotubes and immunoprecipitation of PKRα. L6 cells were differentiated into myotubes on 10 cm diameter dishes (Hundal et al., 1992). The myotubes were deprived of serum overnight in DMEM, washed three times in phosphate free DMEM and incubated for a further 1 h with 5 ml of this medium. The myotubes were then washed twice with phosphate free DMEM and incubated for 4 h with carrier-free [³²P]orthophosphate (1 mCi/ml). Following incubation in the presence or absence of 100 nM wortmannin for 10 min, the myotubes were stimulated for 5 min at 37° C. in the presence or absence of 100 nM insulin and placed on ice, The medium was aspirated, the myotubes washed twice with ice-cold DMEM buffer and then lysed with 1.0 ml of ice-cold Buffer A (50 mM Tris/HCl pH 7.5,1 mM EDTA 1 mM EGTA, 1% (by vol) Triton X100, 1 mM sodium orthopervanadate, 10 mM sodium glycerophosphate, 50 mM NaF, 5 mM sodium pyrophosphate, 1 μM Microcystin-LR, 0.27 M sucrose, 1 mM benzamidine, 0.2 mM phenylmethylsulphonyl fluoride, 10 μg/ml leupeptin, and 0.1% (by vol) 2-mercaptoethanol). The lysates were centrifuged at 4° C. for 10 min at 13,000×g and the supernatants incubated for 30 min on a shaking platform with 50 μl of Protein G-Sepharose coupled to 50 μg of preimmune sheep IgG. The suspensions were centrifuged for 2 min at 13,000×g and the supernatants incubated for 60 min with 30 μl of Protein G-Sepharose covalently coupled to 60 μg of PKBa antibody (Harlow and Lane, 1988). The Protein G-Sepharose-antibody-PKBα complex was washed eight times with 1.0 ml of Buffer A containing 0.5 M NaCl, and twice with 50 mM Tris/HCl pH 7.5, 0.1 mM EGTA and 0.1% (by vol) 2-mercaptoethanol (Buffer B).

Assay of immunoprecipitated PKBa and protein determinations. Three aliquots of each immunoprecipitate (each comprising only 5% of the total immunoprecipitated PKBα) were assayed for PKBα activity towards the peptide GRPRTSSFAEG as described previously (Cross et al., 1995). One unit of activity was that amount which catalysed the phosphorylation of 1 nmol of substrate in 1 min. Protein concentrations were determined by the method of Bradford, 1976.

Tryptic digestion of in vivo phosphorylated PKBα. The immunoprecipitated PKBα was added to an equal volume of 2% (by mass) SDS and 2% (by vol) 2-mercaptoethanol, and incubated for 5 min at 100° C., After cooling to room temperature, 4-vinylpyridine was added to a final concentration of 2% (by vol) and the mixture was incubated for 1 h at 30° C. on a shaking platform, followed by electrophoresis on a 10% polyacrylamide gel. After autoradiography, the 60 kDa band corresponding to rat PKBα was excised and the gel piece homogenized in five vols of 25 mM N-ethylmorpholine HCl, pH 7.7, containing 0.1% (by mass) SDS and 5% (by vol) 2-mercaptoethanol. The suspension was incubated for 1 h at 37° C. on a shaking platform, then centrifuged for 1 min at 13,000×g and the supernatant collected. The pellet was incubated for a further 1 h with five vols of the same buffer and centrifuged for 1 min at 13,000×g. The two supernatants (containing 80-90% of the ³²P-radioactivity) were combined, 0.2 vols of 100% (by mass) trichloroacetic acid added, and the sample incubated for 1 h on ice. The suspension was centrifuged for 10 min at 13,000×g, the supernatant discarded and the pellet washed five times with 0.2 ml of water. The pellet was then incubated at 30° C. with 0.3 ml of 50 mM Tris/HCl pH 8.0, 0.1% (by vol) Triton X100 containing 1 μg of alkylated trypsin. After 3 h another 1 μg of trypsin was added and the suspension left for a further 12 h. Guanidinium hydrochloride (8 M) was added to bring the final concentration to 1.0 M in order to precipitate any residual SDS and, after standing on ice for 10 min, the suspension was centrifuged for 5 min at 13,000×g. The supernatant containing 90% of the ³²P-radioactivity was chromatographed on a Vydac C18 column as described in the legend to FIG. 2.

Transfection of 293 cells and immunoprecipitation of HA-tagged PKBα. Human embryonic kidney 293 cells were cultured at 37° C. in an atmosphere of 5% CO2, on 10 cm diameter dishes in DMEM containing 10% foetal calf serum. Cells were split to a density of 2×106 per 10 cm dish, and after 24 h at 37° C. the medium was aspirated and 10 ml of freshly prepared DMEM containing 10% foetal calf serum added. Cells were transfected by a modified calcium phosphate method (Chen and Okayama, 1988) with lug/ml DNA per plate. 10 μg of plasmid DNA in 0.45 ml of sterile water was added to 50 μl of sterile 2.5 M CaC12, and then 0.5 ml of a sterile buffer composed of 50 mM N,N-bis[2-hydroxyethyl]-2-aminoethanesulphonic acid/HCl pH 6.96, 0.28 M Nacl and 1.5 mM Na2HPO4 was added. The resulting mixture was vortexed for 1 min, allowed to stand at room temperature for 20 min, and then added dropwise to a 10 cm dish of 293 cells). The cells were placed in an atmosphere of 3% CO2, for 16 h at 37° C., then the medium was aspirated, and replaced with fresh DMEM containing 10% foetal calf serum. The cells were incubated for 12 h at 37° C. in an atmosphere of 5% CO2, and then for 12 h in DMEM in the absence of serum. Cells were preincubated for 10 min in the presence of 0.1% DMSO or 100 nM wortmannin in 0.1% DMSO and then stimulated for 10 min with either 100 nM insulin or 50 ng/ml IGF-1 in the continued presence of wortmannin. After washing twice with ice cold DMEM the cells were lysed in 1.0 ml of icecold Buffer A, the lysate was centrifuged at 4° C. for 10 min at 13,000×g and an aliquot of the supernatant (10 μg protein) was incubated for 60 min on a shaking platform with 5 μl of protein G-Sepharose coupled to 2 μg of HA monoclonal antibody. The suspension was centrifuged for 1 min at 13,000×g, the Protein G-Sepharose-antibody-HA-PKBα complex washed twice with 1.0 ml of Buffer A containing 0.5 M NaCl, and twice with Buffer B, and the immunoprecipitate assayed for PKBα activity as described above.

³²P-Labelling of 293 cells transfected with HA-PKBα. 293 cells transfected with HA-PKBα DNA constructs. were washed with phosphate free DMEM, incubated with [32 p] orthophosphate (1 mCi/ml) as described for L6 myotubes, then stimulated with insulin or IGF1 and lysed, and PKBα immunoprecipitated as described above. The NP-labelled HA-PKBα immunoprecipitates were washed, alkylated with 4-vinylpyridine, electrophoresed and digested with trypsin as described above for the endogenous PKBα present in rat L6 myotubes.

Transfection of COS-1 cells and immunoprecipitation of HA-PKBα. COS-1 cells were maintained in DMEM supplemented with 10% FCS at 370 C. in an atmosphere of 5% CO2,. Cells at 70-80% confluency were transfected by a DEAE-dextran method (Seed & Aruffo, 1987), and 48 hours later serum-starved for 24 hours. Cells were lysed in a buffer containing 50 mM Tris-HCl, pH 7.5, 120 mM NaCl, 1% Nonidet P-40, 25 mM NaF, 40 mM sodium-,β-glycerophosphate, 0.1 mM sodium orthopervanadate, 1 mM EDTA, 1 mM benzamidine, 1 mM phenylmethylsulphonyl fluoride, and lysates centrifuged for 15 min at 13,000×g at 4° C. Supernatants were pre cleared once for 30 min at 4° C. with 0.1 vols of 50% Sepharose 4B/25% Pansorbin (Pharmacia and Calbiochem, respectively) and HA-PKBα immunoprecipitated from 1 mg of extract using the 12CA5 antibody coupled to Protein A Sepharose beads. Immunoprecipitates were washed twice with lysis buffer containing 0.5 M NaCl and once with lysis buffer.

Immunoblotting and quantification of levels of PKAα expression. Cell extracts were resolved by 7.5% SDS-PAGE and transferred to Immobilon membranes (Millipore). Filters were blocked for 30 min in a blocking buffer containing 5% skimmed milk in 1×TBS, 1% Triton X-100 and 0.5% Tween 20, followed by a 2 h incubation with the 12CA5 supernatant 1000-fold diluted in the same buffer. The secondary antibody was alkaline conjugated anti-mouse Ig from goat (Southern Biotechnology Associates, Inc), 1000-fold diluted in the blocking buffer. Detection was performed using AP color development reagents from Bio-Rad according to the manufacturer's instructions. Quantification of levels of PKBα expression was achieved by chemiluminescence, using fluroisothiocyanante-labelled antimouse IgG from goat as the secondary antibody and the Storm 840/860 and ImageQuant software from Molecular Dynamics.

All peptides used to assay PKBα, and TTYADFIASGRTGRRNAIHD (the specific peptide inhibitor of cyclic AMP dependent protein kinase—PKI) were synthesised on an Applied Biosystems 431A peptide synthesizer. Their purity (>95%) was established by HPLC and electrospray mass spectrometry, and their concentrations were determined by guantitative amino acid analysis.

Preparation and assay of PKBα. The construction of cytomegalovirus vectors (pCMV5) of the human haemagglutonin epitope-tagged wild type_(HA-PKBα) was described previously (Alessi et al., 1996). 293 cells grown on 10 cm dishes were transfected with a DNA construct expressing HA-PKBα using a modified calcium phosphate procedure (Alessi et al., 1996). The cells were deprived of serum for 16 h prior to lysis and, where indicated, were stimulated for 10 min in the presence of 50 ng/ml IGF-1 to activate PKBα. The cells were lysed in 1.0 ml ice-cold Buffer A (50 mM Tris/HCl pH 7.5, 1 mM EDTA 1 mM EGTA, 1% (by vol) Triton X-100, 1 mM sodium orthovanadate, 10 mM sodium β-glycerophosphate, 50 mM NaF, 5 mM sodium pyrophosphate, 1 μM Microcystin-LR, 0.27 M sucrose, 1 mM benzamidine, 0.2 mM phenylmethylsulphonyl fluoride, 10 μg/ml leupeptin, and 0.1% (by vol) 2-mercaptoethanol) the lysate centrifuged at 4° C. for 10 min at 13,000×g and the supernatant obtained from one 10 cm dish of cells (2-3 mg protein) was incubated for 60 min on a shaking platform with 20 p1 of protein G-Sepharose coupled to 10 μg of HA monoclonal antibody. The suspension was centrifuged for 1 min at 13,000×g, the Protein G-Sepharose-antibody-HA-PKBα complex washed twice with 1.0 ml of Buffer A containing 0.5 M NaCl, and twice with Buffer B (50 mM Tris/HCl pH 7.5, 0.1 mM EGTA, 0.01% (by vol) Brij-35 and 0.1% (by vol) 2-mercaptoethanol). The PKBα immunoprecipitates were diluted in Buffer B to an activity of 2.0 U/ml towards the Crosstide peptide GRPRTSSFAEG and 0.1 ml aliquots snap frozen in liquid nitrogen and stored at −80° C. No significant loss of PKBα activity occurred upon thawing the PKBα immunoprecipitates or during storage at −80° C. for up to 3 months . The standard PKBα_assay (50 μl) contained: 50 mM Tris/HCl pH 7.5, 0.1 mM EGTA, 0.1% (by vol) 2-mercaptoethanol, 2.5 μM PKI, 0.2 U/ml PKBα, Crosstide (30 μM), 10 mM magnesium acetate and 0.1 mM [Y³²P]ATP (100-200 cpm/pmol). The assays were carried out for 15 min at 30° C., the assay tubes being agitated continuously to keep the immunoprecipitate in suspension, then terminated and analysed as described (Alessi et al., 1995). One unit of activity was that amount of enzyme which catalysed the phosphorylation of 1 nmol of Crosstide in 1 min. The phosphorylation of other peptides, histone H2B and MBP were carried out in an identical manner. All the Crosstide activity in HA-PKBα immunoprecipitates is catalysed by PKBα (see Results) and the PKBα concentration in the immunoprecipitates was estimated by densitometric scanning of Coomassie blue-stained polyacrylamide gels, using bovine serum albumin as a standard. Protein concentrations were determined by the method of Bradford using bovine serum albumin as standard (Bradford et al., 1976). Michaelis constants (Km) and Vmax values were determined from double reciprocal plots of 1/V against 1/S, where V is the initial rate of phosphorylation, and S is the substrate concentration. The standard errors for all reported kinetic constants were within <±20%, and the data is reported as mean values for 3 independent determinations. FIG. 16 shows the results relative to those obtained for unstimulated PKBα.

Tryptic digestion of histone 2B phosphorylated by PKBα. Histone H2B (30 μM) was phosphorylated with 0.2 U/ml HA-PKBα. After 60 min 0.2 vol of 100% (by mass) trichloroacetic acid was added, and the sample incubated for 1 h on ice. The suspension was centrifuged for 10 min at 13,000×g, the supernatant discarded and the pellet washed five times with 0.2 ml of ice cold acetone. The pellet was resuspended in 0.3 ml of 50 mM Tris/HCl pH 8.0, 0.1% (by vol) reduced Triton-X100 containing 2 μg of alkylated trypsin and, after incubation for 16 h at 30° C., the digest was centrifuged for 5 min at 13,000×g. The supernatant, containing 95% of the ³²P-radioactivity, was chromatographed on a Vydac C18 column equilibrated with 0.1% (by vol) trifluoroacetic acid (TFA) in water. With reference to the results shown in FIG. 17, the columns were developed with a linear acetonitrile gradient (diagonal line) at a flow rate of 0.8 ml/min and fractions of 0.4 ml were collected. (A) Tryptic peptide map of ³²P-labelled histone H2B, 70% of the radioactivity applied to the column was recovered from the major ³²P-peptide eluting at 19.5% acetonitrile. (B) A portion of the major ³²P-peptide (50 pmol) was analysed on an Applied Biosystems 476A sequencer, and the phenylthiohydantoin (Pth) amino acids identified after each cycle of Edman degradation are shown using the single letter code for amino acids. A portion of the major ³²P-peptide (1000 cpm) was then coupled covalently to a Sequelon arylamine membrane and analysed on an Applied Biosystems 470A sequencer using the modified programme described in (Stokoe et al., 1992). ³²P radioactivity was measured after each cycle of Edman degradation.

TABLE 7.1
Molecular basis for the substrate specificity of PKBα
The phosphorylated residue is shown in boldface type, the altered residue
is underlined. V(100 μM) is the relative rate of phosphorylation
at 0.1 mM peptide relative to peptide 1. ND, not determined. *An alanine
residue was added to the C-terminal of the peptide RPRTSSP, since we
have experienced difficulty in synthesing peptides terminating in proline.

| | Peptides | Km (μM) | Vmax (U/mg) | V(0.1 mM) |
|---|---|---|---|---|
| A | | | | |
| 1. | GRPRTSSFAEG | 4 | 250 | 100 |
| 2. | RPRTSSFA | 8 | 305 | 109 |
| 3. | GRPRTSSF | 8 | 385 | 129 |
| 4. | RPRTSSF | 5 | 260 | 105 |
| 5. | RPRTSTF | 30 | 243 | 78 |
| 6. | RPRTSAF | — | 0 | |
| 7. | PRTSSF | — | 0 | |
| 8. | RPRTSS | >500 | ND | 2 |
| 9. | KPRTSSF | >500 | ND | 4 |
| 10. | RPKTSSF | >500 | ND | 2 |
| B | | | | |
| 1. | RPRTSSF | 5 | 260 | 105 |
| 2. | RPRTSSL | 8 | 278 | 104 |
| 3. | RPRTSSV | 21 | 300 | 102 |
| 4. | RPRTSSA | 250 | 265 | 30 |
| 5. | RPRTSSK | 80 | 308 | 67 |
| 6. | RPRTSSE | >500 | ND | 9 |
| 7. | RPRTSSPA* | — | 0 | |
| C | | | | |
| 1. | RPRTSSF | 5 | 260 | 105 |
| 2. | RPRASSF | 12 | 230 | 89 |
| 3. | RPRVSSF | 25 | 273 | 77 |
| 4. | RPRGSSF | 60 | 163 | 37 |
| 5. | RPRNSSF | >500 | ND | 21 |
| 6. | RPRTASF | 20 | 213 | 83 |
| 7. | RPRTGSF | 25 | 233 | 77 |
| 8. | RPRTVSF | 30 | 365 | 89 |
| 9. | RPRTNSF | 30 | 300 | 81 |
| 10. | RPRAASF | 25 | 215 | 77 |
| 11. | RFRGGSF | 105 | 345 | 55 |
| 12. | RPRGASF | 105 | 160 | 37 |
| 13. | RPRAGSF | 49 | 114 | 70 |

TABLE71.2
Comparison of the substrate specificities of PKBα, MAPKAP kinase-1,
and p70S6 kinase. Peptides 1 and 2 are very good substrates for MAPKAP
kinase-1 and p70 S6 kinase, and peptide 3 is a relatively specific
substrate for p70 S6 kinase [16].

| | Protein kinase Bα | | MAPKAP kinase-1 | | p70 S6 kinase | |
|---|---|---|---|---|---|---|
| | Kₘ | Vₘₐₓ | Kₘ | Vₘₐₓ | Kₘ | Vₘₐₓ |
| Peptide | (mM) | (U/mg) | (mM) | (U/mg) | (mM) | (U/mg) |
| A | | | | | | |
| 1. KKKNRTLSVA | 185 | 270 | 0.2* | 1550* | 3.3* | 890* |
| 2. KKRNRTLSVA | 80 | 300 | 0.7* | 1800* | 1.5* | 1520* |
| 3. KKRNKTLSVA | >500 | ND | 17* | 840* | 34* | 760* |
| 4. KKRNRTLTV | 388 | 330 | 40* | 270* | 4.8* | 1470* |
| B | | | | | | |
| 1. GRPRTSSFAEG | 4 | 250 | 2 | 790 | 3 | 1270 |
| 2. RPRTSSF | 5 | 260 | 12 | 840 | 125 | 705 |
| 3. RPRTSTF | 30 | 240 | >500 | ND | 211 | 590 |
| 4. RPRAASF | 25 | 215 | 20 | 1020 | >500 | ND |
| 5. RPRAATF | 25 | 230 | >500 | ND | >500 | ND |

*Data reported previously [16]; ND, not determined.

The following documents are incorporated herein by reference.

### REFERENCES

Alessi, D. R., Andjelkovic, M., Caudwell, F. B., Cron, P., Morrice, N. Cohen, P. and Hemmings, B. (1996) EMBO J. 15, 6541-6552.

Alessi, D. R., Cohen, P., Leevers, S., Cowley, S. and Marshall, C. J. (1995) Methods Enzymol 255, 279-290.

Ahmed, N. N., Franke, T. F., Bellacosa, A., Datta, K., Gonzales-Portal, M. E., Taguchi, T., Tesra, J. R. and Tsichlis, P. N. (1995) Mol. Cell. Biol. 15, 2304-2310.

Alessi, D. R., Cuenda, A., Cohen, P., Dudley, D. and Saltiel, A. L. (1995) J. Biol. Chem. 270, 27489-27494.

Andersson, S., Davie, D. N., Dahlback, H., Jornvall, H. Russell, D. W. (1989) J. Biol. Chem. 264, 8222-8229.

Andjelkovic, M., Jones, P. F., Grossniklaus, U., Cron, P., Schier, A. F., Dick, M, Bilbe, G. and Hemmings, B. A. (1995) J. Biol. Chem. 270, 4066-4075.

Andjelkovic, M., Jakubowicz, T., Cron, P., Ming X. F., Han, J. H. and Hemmings, B. A. (1996) Proc. Natl. Acad. Sci. USA, 93, 5699-5704.

Andjelkovic, M., Jakubowicz, T., Cron, P., Ming X. F., Han, J. H. and Hemmings, B. A. (1995) Proc. Natl. Acad. Sci. USA, 93, submitted.

Belacossa, A., Testa, J. R., Staal, S. P. and Tsichlis, P. N. (1991) Science, 254, 244-247.

Ben-Levy, R., Leighton, I. A., 13Doza, Y. N., Attwood, P., Morrice, N., Marshall, C. J. and Cohen, P., (1995) EMBO J. 14, 5920-5930.

Bos, J. L. (1995) Trends Biochem. Sci., 20, 441-442.

Bradford, M. M. (1976) Anal Biochem, 72 248-254.

Burgering, B. M. T. and Coffer, P. J. (1995) Nature, 376, 599-602.

Carpenter, C. L. and Cantley, L. C. (1996) Curr. Opinion Cell Biol.8, 253-158.

Chen, C. And Okayama, H. (1988) Biotechniques 6, 632.

Cheng, J. Q., Godwin, A. K., Bellacosa, A., Taguchi, T., Franke, T. F., Hamilton, T. C., Tsichlis, P. N. and Testa, J. R. (1992) Proc. Natl. Acad. Sci. USA, 89, 9267-9271.

Cheng, J. Q., Ruggeri, B., Klein, W. M., Sonoda, G., Altomare, D. A., Watson, D. K., Testa, J. R.(1996) Proc. Natl. Acad. Sci. U.S.A. 93, 3636-3641.

Coffer, P. J. and Woodgett, J. R. (1991) Eur. J. Biochem., 201, 475-481.

Cross, D. A. E., Alessi, D. R., Vandenheede, J. R., McDowell, H. E., Hundal, H. S. and Cohen, P. (1994) Biochem. J. 303, 21-26.

Cross, D. A. E., Alessi, D. R., Cohen, P. Andjelkovic, M. and Hemming, B. A. (1995) Nature, 378, 785-789.

Cuenda, A., Rouse, J., Doza, Y. N., Meier, R., Young, P. R., cohen, P. and Lee, J. C. (1995) FEBS Lett. 364, 229-233.

Dhand, R., Hils, I., Panayotou, G., Roche, S., Fry, J. M., Gout, I., Totty, N. P., Troung, O., Vicendo, P., Yonezawa, K., Kasuga, M., Courtneidge, S. A., Waterfield, M. D. (1994) EMBO J. 13, 522-533.

Eldar-Finkelman, H., Seger, R., Vandenheede, J. R. & Krebs, E. G. (1995) J.Biol.Chem. 270, 987-990.

Embi, N., Rylatt, D. B. and Cohen, P. (1980) Eur. J. Biochem. 107, 519-527.

Fiol, C., Williams, J., Chou, C-H., Wang, M., Roach, P. and Andrisani, O. (1994) J. Biol. Chem. 269, 32187-32193.

Franke, T. F., Yang, S. I., Chan, T. O., Datta, K., Kazlauskas, A., Morrison, D. K., Kaplan, D. R. and Tsichlis, P. N. (1995) Cell, 81, 727-736.

Frech, M., Andjelkovic, M., Falck., J. R. and Hemmings, B. A. (1996) Manuscript in preparation.

Goode, N., Hughes, K., Woodget, J. R. and Parker, P. J. J. (1995) J. Biol. Chem. 270, 22412-22416.

de Groot, R., Anwerx, J., Bourouis, M. and Sassone-Corsi, P. (1993) Oncogene 8, 841-847.

Gould, G. W., Cuenda, A., Thomson, F. J. and Cohen, P. (1995) Biochem. J.311, 735-738.

Harlow, E. and Lane, D.(1988) Antibodies a laboratory manual, Cold Spring Harbor Laboratory.

He, X., Saint-Jenner, J-P., Woodgett, J. R., Varuus, H. E. and Dawid, L. B. (1995) Nature 374, 617-622.

Hughes, K., Ramamkrishna, S., Benjamin, W. B. and Woodgett, J. R (1992) Biochem. J. 288, 309-314.

Hundal, H. S., Ramlal, T., Reyes, R., Leiter, L. A. and Klip, A. (1992) Endocrinology 131, 1165-1171.

Hyvonen, M., Macias, M. J., Nilges, M., Oschkinat, H., Saraste, M. and Wilmanns, M. (1995) EMBO J.14, 4676-4685.

James, S. R., Downes, C. P., Gigg, R., Grove, S. J. A., Holmes, A. B. and Alessi, D. R. (1996) Biochem. J. 315, 709-713.

Jones, P. F., Jakubowicz, T. and Hemmings, B. A. Cell Regul. 2, 1001-1009.

Jones, P. F., Jakubowicz, T., Pitossi, F. J., Maurer, F. and Hemmings, B. A. (1991) Proc. Natl. Acad. Sci U.S. A. 88, 4171-4175.

Konishi, H., Kuroda, S., Tanaka, M., Matsuzaki, H., Ono, Y., Kameyama, K., Haga, T. and Kikkawa (1995) Biochem. Biophys. Res. Comm. 216, 526-534.

Kohn, A. D., Kovacina, K. S. and Roth, R. A. (1995) EMBO J., 14, 4288-4295.

Kuo, C. J., Chung, J., Fiorentio, D. F., Flanagan, W. M., Blenis, J. and Crabtree, G. R. (1992) Nature 358, 70-73.

Lam, X., Carpenter, C. L., Ruderman, N. B., Friel, J. C. and Kelly, K. L. (1994) J. Biol. Chem. 269, 20648-20652.

Lazar, D. F, Brady, J., Mastick, C. C., Waters, S. B., Yamauchi, K., Pessin, J. E., Chatracasas, P. and Saltiel, A. (1995) J. Biol. Chem. 270. 20801-20807.

Leighton, I. A., Dalby, K. N., Caudwell, F. B., Cohen, P. T. W. and Cohen, P. (1995) FEBS Lett 375, 289-293.

Lemmon, M. A., Ferguson, K. M., O'Brien, R., Sigler, P. B. and Schlessinger, J. (1995) Proc. Natl. Acad. Sci. U.S.A. 92,10472-10476.

Nikolaki, E., Coffer, P., Hemelsoet, R., Woodgett, J. and Defize, L. (1993) Oncogene 8, 833-840.

No, S. H., Hunt, H. D., Hortnon, R. M., Pullen, J. K. and Paese L. R. (1989) Gene 77, 51-59 Pearson R. B. et al., (1995) EMBO J. vol 14, 5278-5287.

Palmer, R. H., Dekker, L. V., Woschoki, R., Le Good, J. A. and Parker, P. J. J. (1995) J.Biol.Chem. 270, 22412-22416.

Parker, P. J. J., Candwell, F. B. and Cohen, P. (1983) Eur. J. Biochem. 130, 227-234.

Pearson, R. B. et al., EMBO. J, vol 14, 5278-5287.

Pinna, L. A. and Ruzzene, M. (1996) Biochem.Biophys. Acta. in the Press.

Rouse, J., Cohen, P., Trigon, S., Morange, M., Alonso-Llamazares A., Zamanilo, D., Hunt, T., Nebreda, A. (1994) Cell 78,1027-1037.

Saito, Y., Vandenheede, J. R. and Cohen, P. (1994) Biochem. J. 296, 15-19.

Saito, Y., Vandenheede, J. R. and Cohen, P. (1994) Biochem. J. 303, 27-31.

Seed, B. And Aruffo, A. (1987) Proc. Natl. Acad. Sci. U.S.A. (1987) 84, 3365-3369.

Siegfried, E., Chou, T-B. and Perrimon, N. (1992) Cell 71, 1167-Staal, S. P., Hartley, J. W. and Rowe, W. P. Proc. Natl. Acad. Sci. U.S.A. (1977) 74, 3065-3070.

Stambolic, V. and Woodget, J. R. (1994) Biochem. J. 303, 701-704.

Stokoe, D., Campbell, D. G., Nakielny, S., Hidaka, H., Leevers, S. J., Marshall, C. and Cohen, P. (1992) EMBO J. 11, 3985-3994.

Stokoe, D., Caudwell, F. B., Cohen, P. T. W. and Cohen, P. (1993) Biochem. J. 296, 842849.

Sutherland, C., Campbell, D. G. and Cohen, P. (1993) Eur. J. Biochem. 212, 581-588.

Sutherland, C., Leighton, I. A. & Cohen, P. (1993) Biochem.J. 296, 15-19.

Sutherland, C. and Cohen, P. (1994) FEBS Lett. 338, 37-42.

Sperber, B. R., Leight, S., Goedert, M. and Lee, V. M. Y. (1995) FEBS Lett 197,159-153.

Tsutakawa, S. E., Medzihradszky, K. F., Flint, A. J., Burlingame A. L. and Koshland, D. E. (1995) J. Biol.Chem. 270, 26807-26812.

Welsh, G. I., Foulstone, E. J., Young, S. J., Tavare, J. M. and Proud, C. G. (1994) Biochem J. 303,15-20.

Welsh, G. I. and Proud, C. G. (1993) Biochem. J. 294, 625-629.

1 The use of a composition of PKB, its analogues, isoforms, inhibitors, activators and/or the functional equivalents thereof, to regulate glycogen metabolism and/or protein synthesis.

2 The use of a composition of PKB, its analogues, isoforms, inhibitors, activators and/or the functional equivalents thereof, for the manufacture of a medicament to regulate glycogen metabolism and/or protein synthesis.

3 The use as claimed in claim 1 or claim 2, to combat disease states where glycogen metabolism and/or protein synthesis exhibits abnormality.

4 The use as claimed in claim 1, 2 or 3, to combat diabetes.

5 The use as claimed in any preceding claim, to combat cancer.

6 The use as claimed in claim 5, wherein the cancer is breast, pancreatic or ovarian cancer.

7 The use as claimed in any preceding claim, wherein the PKB is PKBα, β or γ, an analogue, isoform, inhibitor, activator or a functional equivalent thereof.

8 The use as claimed in any preceding claim, wherein the PKB, its analogue, isoform, or functional equivalent is modified at one or both of amino acids 308 and 473 by phosphorylation and/or mutation.

9 A composition of PKB, its analogues, isoforms, inhibitors, activators and/or the functional equivalents thereof.

10 A peptide having or including the amino acid sequence Arg-Xaa-Arg-Yaa-Zaa-Ser/Thr-Hyd, where Xaa is any amino acid, Yaa and Zaa are any amino acid, and Hyd is a large hydrophobic residue, or a functional equivalent of such a peptide.

11 A peptide as claimed in claim 10, wherein Hyd is Phe or Leu, or a functional equivalent thereof.

12 A peptide as claimed in claim 10 or claim 11, wherein Yaa or Zaa or both are an amino acid other than glycine.

13 A peptide as claimed in claim 10, having the amino acid sequence GRPRTSSFAEG, or a functional equivalent thereof.

14 A method of identifying agents able to influence the activity of GSK3, said method comprising:
a. exposing a test substance to a substrate of GSK3; and
b. detecting whether said substrate has been phosphorylated.

15 A method of identifying agents which influence the activity of PKB, comprising:
a. exposing a test substance to a sample containing PKB, to form a mixture;
b. exposing said mixture to a peptide as claimed in claim 10, 11, 12 or 13; and
c. detecting whether (and, optionally, to what extent) said peptide has been phosphorylated.

16 A method as claimed in claim 14 or 15, wherein the extent of phosphorylation of the peptide is determined.

17 A method as claimed in claim 15, wherein the phosphorylation state(s) of one or both of amino acids 308 and 473 on PKB is determined.

18 A method as claimed in any one of claims 14 to 17, wherein the test substance is an analogue, isoform, inhibitor, or activator of PKB.

19 A method as claimed in any one of claims 14 to 18, wherein steps a or b (or both) are carried out in the presence of divalent cations and ATP.

20 A method of treatment of the human or non-human animal body, said method comprising administering PKB, its analogues, inhibitors, stimulators or functional equivalents thereof to said body.

21 A method as claimed in claim 20, to combat disease states where glycogen metabolism and/or protein synthesis exhibits abnormality.

22 A method as claimed in claim 20 or 21, to combat diabetes.

23 A method as claimed in claim 20 or 22, to combat cancer.

24 A method as claimed in claim 23, wherein the cancer is breast, pancreatic or ovarian cancer.

25 A method as claimed in any one of claims 20 to 24, wherein the PKB is PKBα, β or γ, an analogue, isoform, inhibitor, activator or a functional equivalent thereof.

26 An agent capable of influencing the activity of PKB, its isoforms, analogues and/or functional equivalents, by modifying amino acids 308 and/or 473 by phosphorylation or mutation.

27 A method of determining the ability of a substance to affect the activity or activation of PKB, the method comprising exposing the substance to PKB and phosphatidyl inositol polyphosphate and determining the interaction between PKB and the phosphatidyl inositol polyphosphate.

28 A method of determining the ability of a substance to combat diabetes, cancer, or any disorder which involves irregularity of protein synthesis or glycogen metabolism, the method comprising exposing the substance to PKB and phosphatidyl inositol polyphosphate and determining the interaction between PKB and the phosphatidyl inositol polyphosphate.

29 A method as claimed in claim 27 or claim 28, wherein the interaction between PKB and the phosphatidyl inositol polyphosphate is measured by assessing the phosphorylation state of PKB.

30 A method as claimed in claim 29, wherein the phosphorylation state of PKB at T308 and/or S473 is assessed.

31 A method of identifying activators or inhibitors of GSK3 comprising exposing the substance to be tested to GSK3 and determining the state of activation of GSK3.

32 A method as claimed in claim 31 wherein the state of activation of GSK3 is determined by assessing its phosphorylation.

33 A method of determining the suitability of a test substance for use in combatting diabetes, cancer, or any disorder which involves irregularity of protein synthesis or glycogen metabolism, the method comprising exposing the substance to be tested to GSK3 and determining the state of activation of GSK3.

34 A method for screening for inhibitors or activators of enzymes that catalyse the phosphorylation of PKB, the method comprising exposing the substance to be tested to
one or more enzymes upstream of PKB;
PKB; and (optionally)
nucleoside triphosphate and determining whether (and optionally to what extent) the PKB has been phosphorylated on T308 and/or S473.
